(19) United States
(12) Patent Application Publication
(10) Pub. No.: US 2024/0408080 A1
(43) Pub. Date: Dec. 12, 2024

(54) PHARMACEUTICAL COMPOSITIONS OF CGRP INHIBITORS AND METHODS OF THEIR USE

(71) Applicant: Pfizer Ireland Pharmaceuticals

(72) Inventors: Vladimir Coric, Madison, CT (US); Charles M. Conway, Cheshire, CT (US); Robert Croop, Newton Square, PA (US); Rajesh Kumar, Skillman, NJ (US)

(21) Appl. No.: 18/262,791

(22) PCT Filed: Jan. 31, 2022
(86) PCT No.: PCT/US2022/014476

§ 371 Date: Jul. 25, 2023

Related U.S. Application Data
(60) Provisional application No. 63/143,980, filed on Feb. 1, 2021.

(51) Int. Cl. A61K 31/496; A61K 9/00; A61P 25/06

(52) U.S. Cl. CPC A61K 31/496; A61K 9/0043; A61P 25/06

(57) ABSTRACT

Provided is pharmaceutical composition for treating a pain disorder in a subject in need thereof, wherein the pharmaceutical composition includes a therapeutically active ingredient including an intranasally bioavailable CGRP inhibitor. Also provided is a method of treating a pain disorder in a subject in need thereof, wherein the method includes intranasally administering to the subject a composition including a therapeutically active component including an intranasally bioavailable CGRP inhibitor.

### CROSS-REFERENCE TO RELATED APPLICATIONS

This application claims priority to U.S. Provisional Application No. 63/143,980 filed Feb. 1, 2021, and all the benefits accruing therefrom under 35 U.S.C. § 119, the contents of which is incorporated herein in its entirety by reference.

### FIELD OF THE INVENTION

The present invention relates to intranasal pharmaceutical compositions of calcitonin gene-related peptide (CGRP) antagonists and methods of their delivery. The compositions and methods may be used for treating CGRP-related disorders such as pain.

### BACKGROUND OF THE INVENTION

Migraine is a chronic and debilitating disorder characterized by recurrent attacks lasting four to 72 hours with multiple symptoms, including typically one-sided, pulsating headaches of moderate to severe pain intensity that are associated with nausea or vomiting, and/or sensitivity to sound (phonophobia) and sensitivity to light (photophobia). Migraines are often preceded by transient neurological warning symptoms, known as auras, which typically involve visual disturbances such as flashing lights, but may also involve numbness or tingling in parts of the body. Migraine is both widespread and disabling. The Migraine Research Foundation ranks migraine as the world's third most prevalent illness, and the Global Burden of Disease Study 2015 rates migraine as the seventh highest specific cause of disability worldwide. According to the Migraine Research Foundation, in the United States, approximately 36 million individuals suffer from migraine attacks. While most sufferers experience migraine attacks once or twice per month, more than 4 million people have chronic migraine, defined as experiencing at least 15 headache days per month, of which at least eight are migraine, for more than three months. Others have episodic migraine, which is characterized by experiencing less than 15 migraine days per month. People with episodic migraine may progress to chronic migraine over time. Migraine attacks can last four hours or up to three days. More than 90% of individuals suffering from migraine attacks are unable to work or function normally during a migraine attack, with many experiencing comorbid conditions such as depression, anxiety and insomnia. Also, those suffering from migraine often have accompanying nausea and have an aversion to consuming food or liquids during an attack.

CGRP (calcitonin gene-related peptide) is a 37 amino acid neuropeptide, which belongs to a family of peptides that includes calcitonin, adrenomedullin and amylin. In humans, two forms of CGRP (α-CGRP and R-CGRP) exist and have similar activities. They vary by three amino acids and exhibit differential distribution. At least two CGRP receptor subtypes may also account for differential activities. The CGRP receptor is located within pain-signaling pathways, intracranial arteries and mast cells and its activation is thought to play a causal role in migraine pathophysiology. For example, research and clinical studies have shown: serum levels of CGRP are elevated during migraine attacks, infusion of intravenous CGRP produces persistent pain in migraine sufferers and non-migraine sufferers, and treatment with anti-migraine drugs normalizes CGRP activity.

Currently, clinicians use a number of pharmacologic agents for the acute treatment of migraine. A study published by the American Headache Society in 2015 concluded that the medications deemed effective for the acute treatment of migraine fell into the following classes: triptans, ergotamine derivatives, non-steroidal anti-inflammatory drugs (“NSAIDs”), opioids and combination medications. The current standard of care for the acute treatment of migraine is prescription of triptans, which are serotonin 5-HT 1B/1D receptor agonists. Triptans have been developed and approved for the acute treatment of migraine over the past two decades. The initial introduction of triptans represented a shift toward drugs more selectively targeting the suspected pathophysiology of migraine. While triptans account for almost 80% of anti-migraine therapies prescribed at office visits by healthcare providers, issues such as an incomplete effect or headache recurrence remain important clinical limitations. In fact, only about 30% of patients from clinical trials are pain free at two hours after taking triptans. In addition, triptans are contraindicated in patients with cardiovascular disease, cerebrovascular disease, or significant risk factors for either because of potential systemic and cerebrovascular vasoconstriction from the 5-HT 1B-mediated effects. Also, according to a January 2017 study published in the journal Headache, an estimated 2.6 million migraine sufferers in the United States have a cardiovascular event, condition or procedure that limits the potential of triptans as a treatment option.

Accordingly, there remains a significant unmet medical need for a novel migraine-specific medication that provides enhanced patient benefits compared to existing therapies.

Possible CGRP involvement in migraine has been the basis for the development and clinical testing of a number of compounds, including for example, advanced clinical candidates rimegepant (BHV-3000) and zavegepant (BHV-3500), which are developed by Biohaven Pharmaceutical Holding Company Ltd., New Haven, CT.

Zavegepant (also known as vazegepant) is a third generation, high affinity, selective and structurally unique small molecule CGRP receptor antagonist having the following formula I.

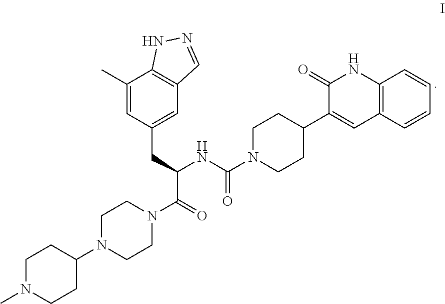 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12]

Zavegepant is described, for example, in WO 03/104236 published Dec. 18, 2003 and U.S. Pat. No. 8,481,546 issued Jul. 9, 2013, which are incorporated herein in their entireties by reference.

While zavegepant is a highly soluble molecule, its bioavailability characteristics may render it challenging to prepare the drug in an oral dosage form. Enhancing the bioavailability of zavegepant and other CGRP inhibitors by different administration routes would therefore be desirable.

Calcitonin gene-related peptide (CGRP) is widely distributed in nociceptive pathways in human peripheral and central nervous system and its receptors are also expressed in pain pathways. While CGRP is involved in migraine pathophysiology, its role in non-headache pain has not been quite clear. There remains a need for new medicines to treat various pain disorders in patients in need thereof.

### SUMMARY OF THE INVENTION

The present invention is directed to the treatment of CGRP related conditions, e.g., migraine or non-migraine-related disorders, by intranasal administration of a pharmaceutical composition including a pharmaceutically active component including a CGRP inhibitor.

In an embodiment, provided is a pharmaceutical composition for treatment or prevention of a pain disorder associated with aberrant levels of CGRP in a subject in need thereof, wherein the pharmaceutical composition includes a therapeutically active component including an intranasally bioavailable CGRP inhibitor.

In another embodiment, provided is apparatus for treatment or prevention of a pain disorder associated with aberrant levels of CGRP in a subject in need thereof including: (a) a reservoir having a sprayable liquid composition including a therapeutically active component including an intranasally bioavailable CGRP inhibitor, (b) an atomization device configured for insertion in a nostril, and (c) means for actuating the device to deliver droplets of the composition to the nostril.

In another embodiment, provided is a method for treatment or prevention of a pain disorder associated with aberrant levels of CGRP in a subject in need thereof, wherein the method includes intranasally administering to the subject a composition including a therapeutically active component including a CGRP inhibitor.

In another embodiment, provided is a kit for treating or preventing a pain disorder in a patient in need thereof, wherein the kit includes: (a) the above pharmaceutical composition for intranasal delivery, and (b) instructions for administering the pharmaceutical composition. The kit may further include an apparatus for administering the pharmaceutical composition.

### BRIEF DESCRIPTION OF THE DRAWINGS

These and/or other aspects will become apparent and more readily appreciated from the following description of the embodiments, taken in conjunction with the accompanying drawings in which:

FIG. 1A is an image of the Aptar Pharma Unidose System for intranasal administration of the composition according to an embodiment;

FIG. 1B is a cross-sectional image of the Aptar Pharma Unidose System for intranasal administration of the composition according to an embodiment;

FIGS. 2A-2F are graphs of mean plasma concentration (nanograms per milliliter, ng/mL) versus nominal time (hour, h) showing plasma concentration levels by day and treatment with the composition according to an embodiment; and

FIG. 3 is a graph showing pain relief in the patients following intranasal administration of zavegepant through 2 hours post-dose.

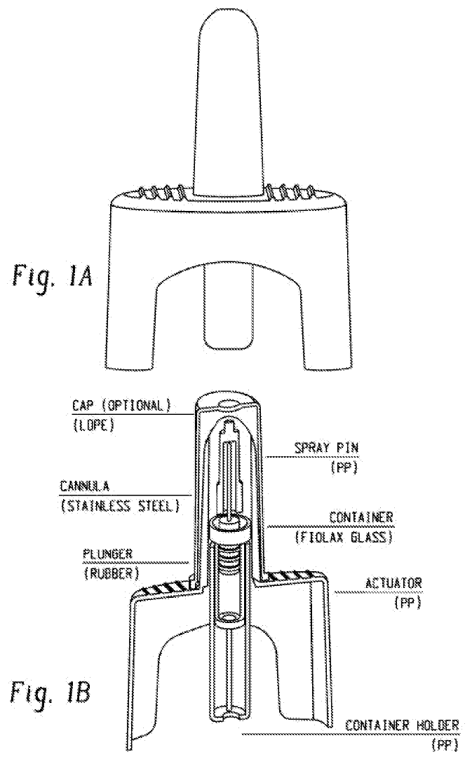

### DETAILED DESCRIPTION OF THE INVENTION

The following detailed description is provided to aid those skilled in the art in practicing the present invention. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting. Terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

The term “about” as used herein refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

The term “administering” as used herein refers to the physical introduction of a composition including a therapeutic agent to a subject, using any of the various methods and delivery systems known to those skilled in the art. Administering can also be performed, for example, once, a plurality of times, and/or over one or more extended periods and can be a therapeutically effective dose or a subtherapeutic dose.

The term “AUC” (area under the curve) as used herein refers to a total amount of drug absorbed or exposed to a subject. Generally, AUC may be obtained from mathematical method in a plot of drug concentration in the subject over time until the concentration is negligible. The term “AUC” (area under the curve) could also refer to partial AUC at specified time intervals.

The term “AUC_{[0-4]}” as used herein refers to area under the concentration-time curve from time 0 to the last measurable concentration.

The term “AUC_{[0-inf]}” as used herein refers to area under the concentration-time curve from time 0 to infinity.

The term “Cₘₐₓ” as used herein refers to a maximum concentration of a drug in blood, serum, a specified compartment or test area of a subject between administration of a first dose and administration of a second dose. The term Cₘₐₓ could also refer to dose normalized ratios if specified.

The terms “in combination with” as used herein refer to administration of one treatment modality in addition to another treatment modality. As such, “in combination with” refers to administration of one treatment modality before, during, or after administration of the other treatment modality to the subject.

The term “pharmaceutically acceptable salt” as used herein refers to a salt form of one or more of the compounds or prodrugs described herein which are presented to increase the solubility of the compound in the gastric or gastroenteric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art.

The terms “subject” and “patient” as used herein refer any human or non-human animal. The term “non-human animal” includes, but is not limited to, vertebrates such as non-human primates, sheep, dogs, and rodents such as mice, rats and guinea pigs. In some embodiments, the subject is a human. The terms, “subject” and “patient” are used interchangeably herein.

The terms “effective amount”, “therapeutically effective amount”, “therapeutically effective dosage” and “therapeutically effective dose” of an agent (also sometimes referred to herein as a “drug”) as used herein refers to any amount of the agent that, when used alone or in combination with another agent, protects a subject against the onset of a disease or promotes disease regression evidenced by a decrease in severity of disease symptoms, an increase in frequency and duration of disease symptom-free periods, or a prevention of impairment or disability due to the disease affliction. The therapeutically effective amount of an agent can be evaluated using a variety of methods known to the skilled practitioner, such as in human subjects during clinical trials, in animal model systems predictive of efficacy in humans, or by assaying the activity of the agent in in vitro assays.

The term “Tₘₐₓ” as used herein refers to a time or period after administration of a drug when the maximum concentration (Cₘₐₓ) is reached in blood, serum, a specified compartment or test area of a subject.

The term “BID” as used herein refers to a twice daily dosing.

The term “CV” as used herein refers to a coefficient of variation.

The term “GM” as used herein refers to a geometric mean.

The term “Kel” as used herein refers to elimination rate constant.

The term “max” as used herein means “maximum,” and the term “min” means “minimum”.

The term “QD” as used herein refers to a once a day dosing.

The term “t_{1/2 el}” as used herein refers to apparent elimination half-life.

The term “treatment” as used herein refers to any treatment of a condition or disease in a subject and may include: (i) preventing the disease or condition from occurring in the subject which may be predisposed to the disease but has not yet been diagnosed as having it; (ii) inhibiting the disease or condition, i.e., arresting its development; relieving the disease or condition, i.e., causing regression of the condition; or (iii) ameliorating or relieving the conditions caused by the disease, i.e., symptoms of the disease. Treatment could be used in combination with other standard therapies or alone. Treatment or “therapy” of a subject also includes any type of intervention or process performed on, or the administration of an agent to, the subject with the objective of reversing, alleviating, ameliorating, inhibiting, slowing down or preventing the onset, progression, development, severity or recurrence of a symptom, complication or condition, or biochemical indicia associated with a disease.

With respect to the CGRP-related disease, “treatment” or treating” is an approach for obtaining beneficial or desired clinical results. For purposes of this invention, beneficial or desired clinical results include, but are not limited to, one or more of the following: curing the disease or disorder, improvement in any aspect of a major symptom including lessening severity, alleviation of major symptom intensity, and other associated symptoms, reducing frequency of recurrence, increasing the quality of life of those suffering from the symptom, and decreasing dose of other medications required to treat the symptom.

The term “intranasally bioavailable CGRP inhibitor” as used herein refers to a CGRP inhibitor having bioavailability of 1% or greater, 2% or greater, 3% or greater, 4% or greater, 5% or greater, 10% or greater, 15% or greater, 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, or 95% or greater, following intranasal administration.

The term “small molecule” as used herein refers to a molecule having molar mass of 1000 g/mol or less, 950 g/mol or less, 900 g/mol or less, 850 g/mol or less, 800 g/mol or less, 750 g/mol or less, 700 g/mol or less, 650 g/mol or less, 600 g/mol or less, 550 g/mol or less, 500 g/mol or less, 450 g/mol or less, 400 g/mol or less, 350 g/mol or less, 300 g/mol or less, 250 g/mol or less, or 200 g/mol or less.

The invention encompasses compositions for intranasal administration that include an intranasally bioavailable CGRP inhibitor. The invention further encompasses methods for modulating CGRP and treating patients with medical conditions associated with aberrant levels of CGRP or CGRP receptor signaling by intranasally administering the composition.

As used herein, the term “CGRP inhibitor” refers to a chemical entity that may be an inhibitor of a CGRP ligand or CGRP receptor. Thus, the term “CGRP inhibitor” encompasses CGRP receptor inhibitors. The CGRP inhibitor may be a CGRP inhibitor or CGRP receptor inhibitor. CGRP (calcitonin gene-related peptide) is a 37 amino acid neuropeptide, which belongs to a family of peptides that includes calcitonin, adrenomedullin and amylin. Substantial evidence has been collected to show that CGRP is implicated in pathophysiology of migraine. Clinical trials were carried out to prove that CGRP inhibitors are effective for treating migraine.

The CGRP inhibitor may be a CGRP antibody, a CGRP receptor antibody, an antigen-binding fragment from a CGRP antibody or a CGRP receptor antibody, a CGRP infusion inhibitory protein, a CGRP bio-neutralizing agent, a small molecule CGRP receptor antagonist, a small molecule CGRP inhibitor, or a polypeptide CGRP inhibitor. For example, CGRP inhibitor may be a small molecule CGRP receptor antagonist.

An intranasally bioavailable CGRP inhibitor may be included in the composition in all pharmaceutically acceptable salt forms. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

The invention is intended to include all isotopes of atoms occurring in the CGRP inhibitor. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

The therapeutically active component may include two or more compounds, each of which may be an intranasally bioavailable active pharmaceutical ingredient (“API”), for example, an anti-migraine drug.

The pharmaceutical composition is adapted for intranasal administration. This means that the composition is in a form physically suitable for intranasal delivery of a therapeutically active component. In an embodiment, the composition is in the form of a sprayable liquid. In other embodiments, the composition is in a semi-solid form, for example, a cream, a gel or an ointment. Without being held to a particular theory, it is believed that most of the absorption of a CGRP inhibitor when administered intranasally is through the nasal mucosa.

According to some embodiments, the CGRP inhibitor may be present in the composition at a concentration of at least about 1 mg/mL, at least about 2 mg/mL at least about 3 mg/mL, at least about 4 mg/mL, at least about 5 mg/mL, at least about 10 mg/mL, at least about 15 mg/mL, at least about 20 mg/mL, at least about 25 mg/mL, at least about 30 mg/mL, at least about 35 mg/mL, at least about 45 mg/mL, at least about 50 mg/mL, at least about 55 mg/mL, at least about 60 mg/mL, at least about 65 mg/mL, at least about 70 mg/mL, at least about 75 mg/mL, at least about 80 mg/mL, at least about 85 mg/mL, at least about 90 mg/mL, at least about 95 mg/mL, at least about 100 mg/mL, at least about 125 mg/mL, at least about 150 mg/mL, at least about 175 mg/mL, or at least about 200 mg/mL. A concentration of the CGRP inhibitor may range between any of the above values. For example, the CGRP inhibitor may be present at a concentration of about 1 to about 200 mg/mL, about 2 to about 100 mg/mL, about 5 to about 100 mg/mL, or about 5 to about 50 mg/mL.

The CGRP inhibitor may be administered at a dose of about 1-1000 mg per day. For example, the CGRP inhibitor may be administered at a dose of about 1, 5, 10, 15, 20, 25, 30, 40, 50, 60, 70, 80, 90, 100, 200, 250, 300, 400, 500, 750, or 1000 mg per day. The daily dose of the CGRP inhibitor may range between any of the above values. The composition including a CGRP inhibitor may be administered as a single dose.

The CGRP inhibitor may be administered for at least one week and for as long as needed. For example, the CGRP inhibitor may be administered for one week, two weeks, three weeks, four weeks, five weeks, six weeks, seven weeks, eight weeks, nine weeks, ten weeks, eleven weeks, or twelve weeks.

As used herein, the phrase “an amount of the composition intranasally administrable as a single dose” means a total volume of the composition that can suitably be administered to one or both nostrils of a human or non-human subject to provide a single dose of CGRP inhibitor. Such an amount is a practical volume; not so small as to be incapable of administration by any known device, but not so great that a substantial portion of the dose is not retained in the nostrils. For example, with respect to a sprayable formulation intended for administration to a human subject in two aliquots, one to each nostril, a volume of about 0.05 to about 0.25 mL can suitably be administered to each nostril, for a total amount of about 0.1 mL to about 0.5 mL per dose. It is generally desirable to administer as low a volume as practicable, to reduce any tendency for the composition to be partially lost by drainage through the nasopharyngeal passage. Thus, particularly suitable volumes are typically about 0.05 to about 0.15 mL per nostril. If desired, however, an entire dose can be administered to one nostril.

As will be clear from the disclosure herein, the pharmaceutical composition is useful for administration to subjects of any mammalian species, particularly to human subjects.

The composition may include a solubilizing agent. The solubilizing agent may include a solvent or solvent system for CGRP inhibitor, and this solvent system, itself including one or more solvents, may form the bulk of the medium in which the CGRP inhibitor is dissolved. Regardless of the nature of the solubilizing agent and whether it includes one or more solvents, a sufficient quantity of the solubilizing agent is present to solubilize essentially all of the CGRP inhibitor. The solubilizing agent must be pharmaceutically acceptable when present in an amount needed to solubilize the CGRP inhibitor. For example, the solubilizing agent should not be toxic to nor cause excessive irritation of tissues lining the nasal cavity. In an embodiment, the solvent may be water, alcohol, or a combination thereof. In another embodiment, the solvent may be water.

The composition optionally further includes a receptivity agent. The term “receptivity agent” herein means an agent that, when included in a pharmaceutical composition administered to a subject, is capable of mitigating an undesirable response to the composition at or in proximity to the locus of administration in or on the subject. Specifically, when the locus of administration is intranasal, such undesirable responses that can be mitigated may include an involuntary or reflex response such as sneezing, excessive nasal drip or irritation of nasal tissues, and/or a cognitive response, such as to unpleasant taste or odor. A cognitive response can include a conscious or subconscious decision to reduce or end use of the composition, and can thus affect patient compliance. A receptivity agent can mitigate one or more such undesirable responses.

In some embodiments, the receptivity agent includes an organoleptic enhancing agent. Illustrative examples of organoleptic enhancing agents include natural and/or synthetic sweeteners, flavorants, aromatics, taste-masking compounds, or combinations thereof.

In some embodiments, an organoleptic enhancing agent included as a receptivity agent includes a sweetener. Illustrative sweeteners include saccharin, aspartame, neotame, cyclamates, glucose, fructose, sucrose, xylitol, tagatose, sucralose, maltitol, isomaltulose, hydrogenated isomaltulose, lactitol, sorbitol, mannitol, trehalose, maltodextrin, polydextrose, glycerin, erythritol, maltol, acesulfame, acesulfame potassium, alitame, neohesperidin dihydrochalcone, stevioside, thaumatin, sugars, or combinations thereof.

In an embodiment, the receptivity agent includes an agent that can inhibit sneezing, i.e., an anti-sternutatory agent.

The pharmaceutical composition optionally further includes one or more pharmaceutically acceptable ingredients, for example, ingredients useful as carriers, preservatives, diluents, stabilizers, pH modulating agents, etc. According to an embodiment, the composition includes at least one preservative. Preservatives can have antimicrobial activity and/or can serve as antioxidants. Illustrative preservatives include but are not limited to butylated hydroxytoluene, butylated hydroxyanisole, or combinations thereof.

Where the composition is formulated in an aqueous medium, it may include one or more tonicity modulating agents, for example in an amount that renders the composition substantially isotonic. For example, a saline solution may form the basis of such a composition.

Also provided is an apparatus for intranasal administration of a CGRP inhibitor. The apparatus may include: (a) a reservoir having a sprayable liquid composition including a therapeutically active component including an intranasally bioavailable CGRP inhibitor, (b) an atomization device configured for insertion in a nostril, and (c) means for actuating the device to deliver droplets of the composition to the nostril.

The atomizing device can be any device capable of generating droplets of the liquid composition when the composition is supplied from the reservoir, so long as the device can be inserted in a nostril. In an embodiment, the device includes a nozzle or constricted passage that, when the liquid composition passes through it under pressure, breaks the liquid up into droplets. Any means known in the art for actuating the atomization device can be employed, for example application of pressure as by squeezing the reservoir or depressing a plunger, or in the case of an electrically operated device, activating a switch.

The range of droplet size produced by the apparatus is dependent upon the physical properties of the composition, for example its viscosity, the nature of the atomization device (e.g., size of a nozzle aperture) and the manner in which the device is actuated to discharge the composition. Droplets should generally not be so fine as to form an inhalable aerosol, but not so coarse as to fail to adhere readily to the nasal mucosa.

Optionally, the apparatus is operable to deliver a metered amount of the composition, for example an amount of about 0.05 to about 0.25 mL, more typically about 0.05 to about 0.15 mL, to a nostril. The apparatus is optionally adjustable to deliver different metered amounts. In some embodiments, the apparatus includes a nasal spray device, or a modification thereof, that is commercially available, such as those sold by Aptar Pharma, which is part of AptarGroup, Inc. (Crystal Lake, Illinois, USA) The apparatus may be a unidose apparatus, a bi-dose apparatus, or a multi-dose apparatus.

Also provided is a method for delivering a CGRP inhibitor to a subject, wherein the method includes intranasally administering to the subject a composition including a therapeutically active component including the CGRP inhibitor.

Also provided is a method for treating a condition associated with aberrant levels of CGRP in a subject in need thereof, wherein the method includes intranasally administering to the subject a therapeutically effective amount of a composition including a therapeutically active component including a CGRP inhibitor.

In an embodiment, the condition may be a disease or disorder that is selected from acute migraine, chronic migraine, cluster headache, chronic tension type headache, medication overuse headache, post-traumatic headache, post-concussion syndrome, brain trauma, and vertigo.

In another embodiment, the condition may be a disease or disorder that is selected from chronic pain, neurogenic vasodilation, neurogenic inflammation, inflammatory pain, neuropathic pain, diabetic peripheral neuropathic pain, small fiber neuropathic pain, Morton's neuroma, chronic knee pain, chronic back pain, chronic hip pain, chronic finger pain, exercise-induced muscle pain, cancer pain, chronic inflammatory skin pain, pain from burns, pain from scars, complex regional pain syndrome, burning mouth syndrome, alcoholic polyneuropathy, chronic inflammatory demyelinating polyradiculoneuropathy, human immunodeficiency virus (HIV) or acquired immunodeficiency syndrome (AIDS)-associated neuropathy, drug-induced neuropathy, industrial neuropathy, lymphomatous neuropathy, myelomatous neuropathy, multi-focal motor neuropathy, chronic idiopathic sensory neuropathy, carcinomatous, neuropathy, acute pain autonomic neuropathy, compressive neuropathy, vasculitic/ischaemic neuropathy, tempero-mandibular joint pain, post-herpetic neuralgia, trigeminal neuralgia, eye pain, and tooth pain.

In an example, the condition may be medication overuse headache (MOH), and the subject having the condition may be undergoing treatment for pain, wherein the treatment for pain may include a medicament selected from acute pain medications and chronic pain medications. For example, the treatment for pain includes a medicament selected from triptans, ergot alkaloids, analgesics and opioids. The triptans may be selected from rizatriptan, sumatriptan, naratriptan, eletriptan, donitriptan, almotriptan, frovatriptan, avitriptan, and zolmitriptan. The ergot alkaloids may be selected from clavines, lysergic acid amides and ergopeptines. The ergot alkaloid may also be selected from ergonovine, methylergonovine, methysergide, ergotamine, dihydroergotamine, bromocriptine, ergoloid mesylates and lysergic acid diethylamide, or a combination thereof.

The MOH may result from the chronic use of one or more pain medications. The subject may have a primary headache disorder selected from migraine, cluster-type headache, or tension-type headache. The subject may be currently undergoing treatment or may have received treatment for the primary headache disorder.

The treatment for pain may include a medicament selected from aspirin, diclofenac; diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, piroxicam, salsalate, sulindac, tolmetin, celecoxib, rofecoxib, etoricoxib, valdecoxib, parecoxib, meloxicam, lumiracoxib, or a combination thereof.

The MOH may result from treatment with a medicament selected from ketamine, esketamine, alfentanil, alimemazine, alprazolam, amphetamine, buprenorphine, butorphanol, clonazepam, codeine, cyclobenzaprine, diazepam, dihydrocodeine, dihydromorphine, dronabinol, estazolam, ezopiclone, fentanyl, flurazepam, hydrocodone, hydromorphone, lorazepam, methobarbital, methylphenidate, methadone, morphine, oxycodone, oxymorphone, phenobarbital, secobarbital, tempazepam, tramadol, triazolam, zaleplon, zopiclone, and zolpidem.

The MOH may result from the chronic use of a medicament selected from alimemazine, alprazolam, amphetamine, buprenorphine, butorphanol, clonazepam, codeine, cyclobenzaprine, diazepam, dihydrocodeine, dihydromorphine, dronabinol, estazolam, ezopiclone, fentanyl, flurazepam, hydrocodone, hydromorphone, lorazepam, methobarbital, methylphenidate, methadone, morphine, oxycodone, oxymorphone, phenobarbital, secobarbital, tempazepam, tramadol, triazolam, zaleplon, zopiclone, and zolpidem.

The MOH may result from the chronic use of a medicament selected from aspirin, ibuprofen, naproxen, acetaminophen, diclofenac, flurbiprofen, meclofenamate, isometheptene, indomethacin; codeine, morphine, hydrocodone, acetyldihydrocodeine, oxycodone, oxymorphone, papaverine, fentanyl, alfentanil, sufentanil, remifentanyl, tramadol, prochlorperazine, celecoxib, rofecoxib, meloxicam, piroxicam, JTE-522, L-745,337, NS388, deracoxib, valdecoxib, iumiracoxib, etoricoxib, parecoxib, 4-(4-cyclohexyl-2-methyloxazol-5-yl)-2 fluorobenzenesulfonamide, (2-(3,5-difluorophenyl)-3-(4-(methylsulfonyl)phenyl)-2 cyclopenten-1-one, N-[2-(cyclohexyloxy)-4-nitrophenyl]methanesulfonamide, 2-(3,4 difluorophenyl)-4-(3-hydroxy-3-methylbutoxy)-5-[4-(methylsulfonyl)phenyl]-3(2H)pyridazinone, 2-[(2,4-dichloro-6-methylphenyl)amino]-5-ethyl-benzeneacetic acid, (3Z) 3-[(4-chlorophenyl) [4-(methylsulfonyl)phenyl]methylene]dihydro-2(3H)-furanone, (S)-6,8-dichloro-2-(trifluoromethyl)-2H-1-benzopyran-3-carboxylic acid, amobarbital, butalbital, cyclobarbital, pentobarbital, allobarbital, methylphenobarbital, phenobarbital, secobarbital, vinylbital, verapamil, ciltiazem, Nifedipine, lidocaine, tetracaine, prilocaine, bupivicaine, mepivacaine, etidocaine, procaine, benzocaine, phehelzine, isocarboxazid, dichloralphenazone, nimopidine, metoclopramide, capsaicin receptor agonists, captopril, tiospirone, a steroid, caffeine, metoclopramide, domperidone, scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth subsalicylate, buclizine, cinnarizine, cyclizine, diphenidol, dolasetron, domperidone, dronabinol, droperidol, haloperidol, metoclopramide, nabilone, thiethylperazine, trimethobenzemide, and eziopitant, Meclizine, domperidone, ondansetron, tropisetron granisetron dolasetron, hydrodolasetron, palonosetron, alosetron, cilansetron, cisapride, renzapride metoclopramide, galanolactone, phencyclidine, ketamine, dextromethorphan, and isomers, pharmaceutically acceptable salts, esters, conjugates, or prodrugs thereof.

In another example, the condition may be post-traumatic headache (PTH) headache, and the subject having the condition may experience a PTH one, two, three, four, five, six or seven days after a traumatic incident. The traumatic incident may result a concussion or loss of consciousness. The subject may suffers from dizziness, insomnia, poor concentration, memory problems, photophobia, phonophobia, or fatigue, or a combination thereof.

In another embodiment, the condition may be a disease or disorder that is selected from non-insulin dependent diabetes mellitus, vascular disorders, inflammation, arthritis, thermal injury, circulatory shock, sepsis, alcohol withdrawal syndrome, opiate withdrawal syndrome, morphine tolerance, hot flashes in men and women, flushing associated with menopause, allergic dermatitis, psoriasis, encephalitis, ischaemia, stroke, epilepsy, neuroinflammatory disorders, neurodegenerative diseases, skin diseases, neurogenic cutaneous redness, skin rosaceousness, erythema, tinnitus, obesity, inflammatory bowel disease, irritable bowel syndrome, vulvodynia, polycystic ovarian syndrome, uterine fibroids, neurofibromatosis, hepatic fibrosis, renal fibrosis, focal segmental glomerulosclerosis, glomerulonephritis, IgA nephropathy, multiple myeloma, myasthenia gravis, Sjogren's syndrome, osteoarthritis, osteoarthritic degenerative disc disease, temporomandibular joint disorder, whiplash injury, rheumatoid arthritis, and interstitial cystitis. The skin disease may be selected from recurrent herpes, contact hypersensitivity, prurigo nodularis, chronic pruritus, and uremic pruritus.

In another embodiment, the condition may be a disease or disorder that is selected from chronic obstructive pulmonary disease, pulmonary fibrosis, bronchial hyperreactivity, asthma, cystic fibrosis, chronic idiopathic cough, and a toxic injury. The toxic injury may be selected from chlorine gas injury, mustard gas injury, acrolein injury, smoke injury, ozone injury, warfare chemical exposure, and industrial chemical exposure.

In another embodiment, provided is a kit for treating a condition associated with aberrant levels of CGRP in a patient, wherein the kit includes: (a) the above pharmaceutical composition including a therapeutically active component including an intranasally bioavailable CGRP inhibitor, and (b) instructions for administering the pharmaceutical composition. The kit may further include an apparatus for administering the pharmaceutical composition.

In an embodiment, the invention encompasses compositions for intranasal administration that include (R)-N-(3-(7-methyl-1H-indazol-5-yl)-1-(4-(1-methylpiperidin-4-yl)piperazin-1-yl)-1-oxopropan-2-yl)-4-(2-oxo-1,2-dihydroquinolin-3-yl)piperidine-1-carboxamide (BHV-3500, zavegepant, or compound having formula I) as a small molecule CGRP receptor antagonist.

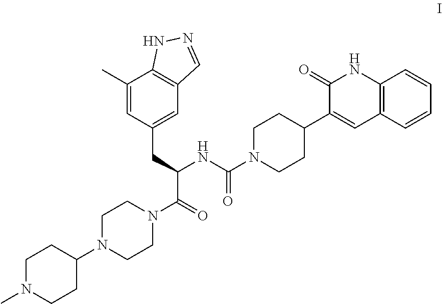 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12]

Zavegepant is also known under an alternative name “vazegepant”, wherein both “zavegepant” and “vazegepant” refer to the same molecule having formula I above.

A method of zavegepant synthesis is described next.

### Synthetic Methods

Abbreviations generally follow conventions used in the art. Chemical abbreviations used in the specification and Examples are defined as follows: “NaHMDS” for sodium bis(trimethylsilyl)amide; “DMF” for N,N-dimethylformamide; “MeOH” for methanol; “NBS” for N-bromosuccinimide; “Ar” for aryl; “TFA” for trifluoroacetic acid; “LAH” for lithium aluminum hydride; “BOC”, “DMSO” for dimethylsulfoxide; “h” for hours; “rt” for room temperature or retention time (context will dictate); “min” for minutes; “EtOAc” for ethyl acetate; “THF” for tetrahydrofuran; “EDTA” for ethylenediaminetetraacetic acid; “Et₂O” for diethyl ether; “DMAP” for 4-dimethylaminopyridine; “DCE” for 1,2-dichloroethane; “ACN” for acetonitrile; “DME” for 1,2-dimethoxyethane; “HOBt” for 1-hydroxybenzotriazole hydrate; “DIEA” for diisopropylethylamine, “Nf” for CF₃(CF₂)₃SO₂—; and “TMOF” for trimethylorthoformate.

Abbreviations as used herein, are defined as follows: “1 x” for once, “2 x” for twice, “3 x” for thrice, “° C.” for degrees Celsius, “eq” for equivalent or equivalents, “g” for gram or grams, “mg” for milligram or milligrams, “L” for liter or liters, “mL” or “ml” for milliliter or milliliters, “μL” for microliter or microliters, “N” for normal, “M” for molar, “mmol” for millimole or millimoles, “min” for minute or minutes, “h” for hour or hours, “rt” for room temperature, “RT” for retention time, “atm” for atmosphere, “psi” for pounds per square inch, “conc.” for concentrate, “sat” or “sat” d “for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry, “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “1H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

Compound I can be prepared according to Scheme 1. This synthesis is 14 chemical steps and highly convergent, coupling the three major fragments in the last three steps. As such, the synthesis begins with the preparation of major fragments A (Scheme 2) and B (Scheme 3).

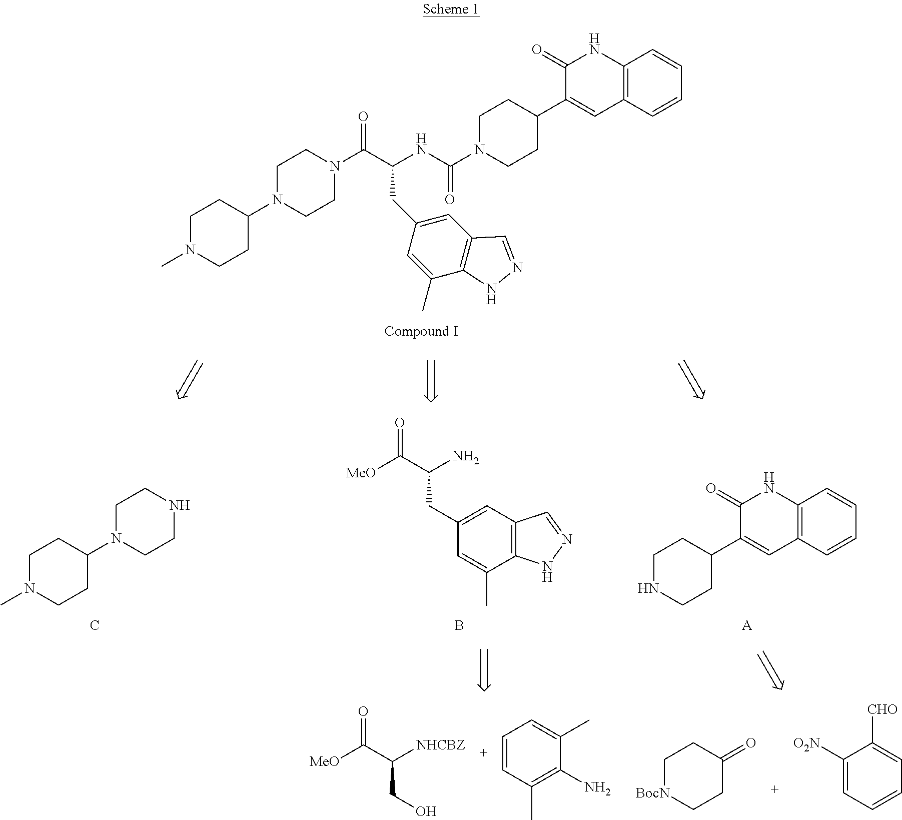

The synthesis of fragment A begins with Horner-Emmons reaction of N-Boc-4-piperidone with the ylide generated from trimethylphosphonoacetate to afford tert-butyl 4-(2-methoxy-2-oxoethylidene)piperidine-1-carboxylate in excellent yield (Scheme 2). Catalytic hydrogenation mediated by palladium on carbon reduces the unsaturated double bond. Treatment of tert-butyl 4-(2-methoxy-2-oxoethyl)piperidine-1-carboxylate with LDA generates the enolate which upon trapping with 2-nitrobenzaldehyde provides the nitro alcohol. Reduction of the nitro group with iron in acetic acid followed by treatment with hydrogen chloride in dioxane completes the synthesis of fragment A.

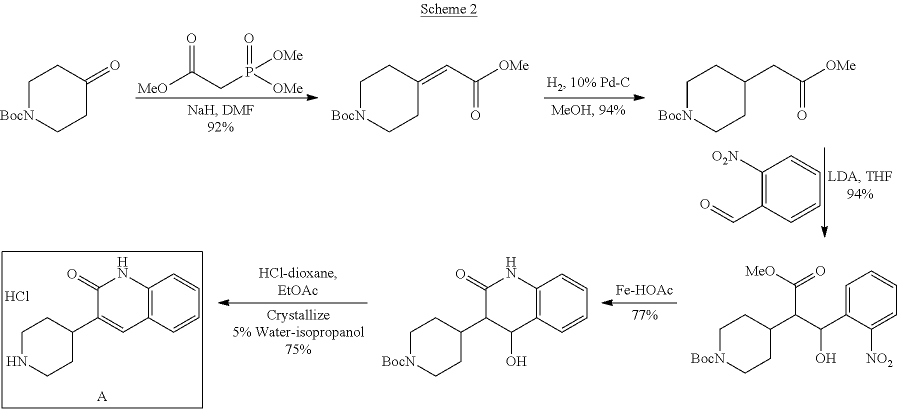

The synthesis of indazole amino acid B begins with the iodination of 2,6-dimethylaniline by the action of iodine monochloride (Scheme 3). This intermediate was temporarily set aside. N-CBZ-L-serine methyl ester undergoes a one-pot methanesulfonylation/elimination reaction to afford N-CBZ-dehydroalanine methyl ester. With the iodide and dehydroalanine in hand, they are efficiently coupled using palladium (II) acetate in a Heck coupling to afford the product in 65% yield.

At this point, the chiral center is installed using a catalytic asymmetric hydrogenation utilizing (−)-1,2-bis((2R,5R)-2,5-diethylphospholano)bezene(cyclooctadiene) rhodium(I) tetrafluoroborate and hydrogen (60 psi) to give the chiral amino acid in ˜₉₆% ee. The indazole ring is then formed by the action of iso-amyl nitrite. The resulting indazole is highly crystalline. One recrystallization from acetone/hexanes affords the indazole amino acid in excellent purity and with an improved 99.8% ee. Removal of the CBZ protecting group under hydrogenation conditions completes the preparation of fragment B.

Indazole amino acid B can also be prepared using enzymatic resolution of the racemic amino acid or keto acid (Hanson, Ronald L.; Davis, Brian L.; Goldberg, Steven L.; Johnston, Robert M.; Parker, William L.; Tully, Thomas P.; Montana, Michael A.; Patel, Ramesh N. Process Research and Development, Bristol-Myers Squibb, New Brunswick, NJ, USA. Organic Process Research & Development (2008), 12(6), 1119-1129.).

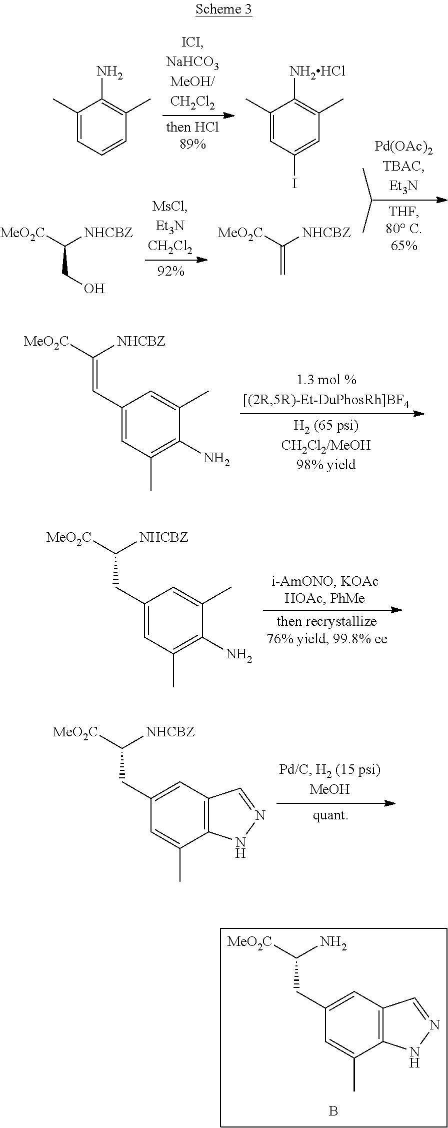

Fragments A and B are efficiently coupled using N,N′-disuccinimidyl carbonate to install the urea moiety in 78% yield (Scheme 4). Saponification of the methyl ester with lithium hydroxide gives a nearly quantitative yield of the carboxylic acid. TBTU© mediated coupling of acid with 1-(1-methylpiperidin-4-yl)piperazine completes the synthesis of Compound I. Flash chromatography affords the product as an amorphous powder which can be crystallized from acetone to afford Compound I as a fine white crystalline powder.

### Scheme 4

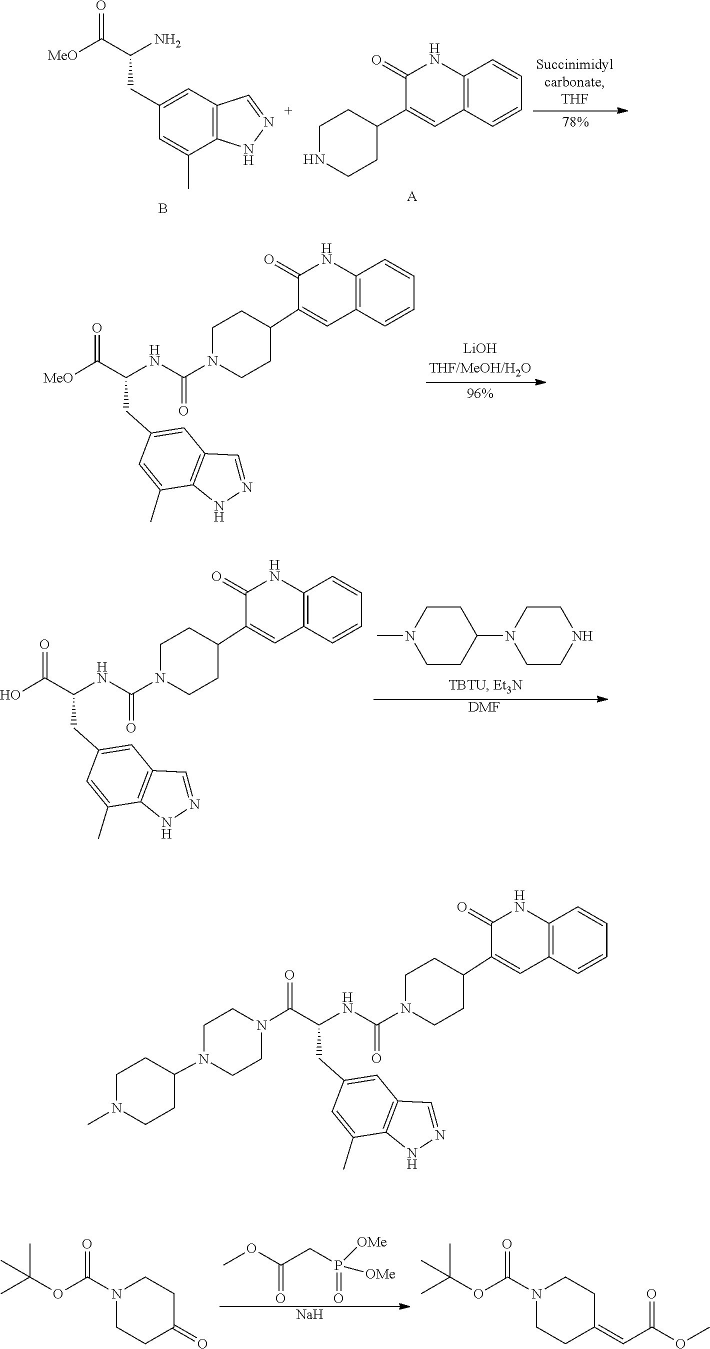

tert-butyl 4-(2-methoxy-2-oxoethylidene)piperidine-1-carboxylate. Sodium hydride in mineral oil (60%, 7.92 g, 198.02 mmoles) was washed with hexanes then suspended in dimethylformamide (220 mL). The mixture was cooled to 0° C. Trimethyl phosphonoacetate (29.0 mL, 189.82 mmoles) was added dropwise to the stirred reaction mixture. After 20 min at 0° C., a solution of N-tert-butoxycarbonyl-4-piperidone (30.41 g, 152.62 mmoles) in dimethylformamide (80 mL) was added to the mixture dropwise. The reaction was stirred at room temperature for 3 h and then diluted with diethyl ether (650 mL). The mixture was washed once with water and the aqueous layer was extracted once with diethyl ether. The combined organic layers were washed 4 times with water and the aqueous phase was discarded. The organic phase was washed with brine and dried over magnesium sulfate, filtered, and concentrated to dryness. The title compound was obtained as a white solid in 92% yield. ¹H-NMR (300 MHz, CDCl₃): δ=5.68 (s, 1H), 3.66 (s, 3H), 3.40-3.51 (m, 4H), 2.90 (t, J=5.49,2 H), 2.25 (t, J=5.49, 2H), 1.44 (s, 9H).

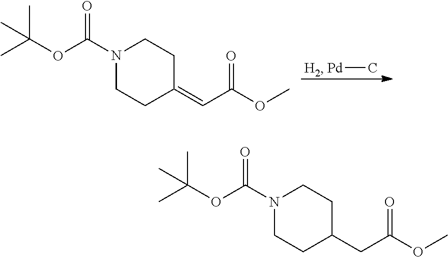

tert-butyl 4-(2-methoxy-2-oxoethyl)piperidine-1-carboxylate. A solution of tert-butyl 4-(2-methoxy-2-oxoethylidene)piperidine-1-carboxylate (35.71 g, 140 mmoles) in a mixture of 1:1 ethyl acetate/methanol (220 mL) was carefully treated with 50% wet 10% palladium on carbon (3.3 g). The reaction vessel was charged with 55 psi of hydrogen gas and the mixture was shaken on a Parr apparatus at room temperature for 16 h. The reaction mixture was then filtered to remove the catalyst and the filtrate concentrated in vacuo. The title compound was obtained as a clear colorless oil in 97% yield. ¹H-NMR (300 MHz, CDCl₃): δ=4.04 (d, J=10.25, 2H), 3.64 (s, 3H), 2.68 (t, J=12.44, 2H), 2.21 (d, J=6.95, 2H), 1.98-1.77 (m, 1H), 1.64 (d, J=13.54, 2H), 1.41 (s, 9H), 1.25-0.99 (m, 2H).

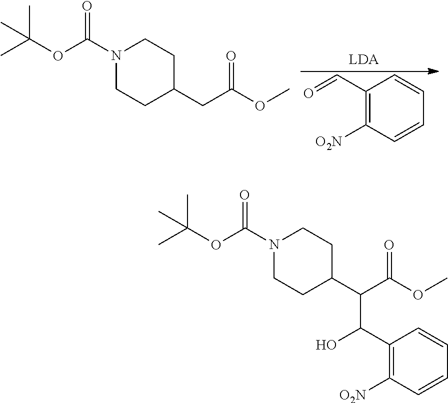

4-[2-Hydroxy-1-methoxycarbonyl-2-(2-nitro-phenyl)-ethyl]-piperidine-1-carboxylic acid tert-butyl ester. N,N-diisopropylamine (4.40 mL, 31.3 mmoles) was dissolved in tetrahydrofuran (50 mL). The mixture was cooled to −78° C. Butyllithium (2.5 M in hexanes, 12.4 mL, 31 mmoles) was added dropwise to the stirred solution. After stirring at −78° C. for 30 min, a solution of tert-butyl 4-(2-methoxy-2-oxoethyl)piperidine-1-carboxylate (6.65 g, 25.8 mmoles) in tetrahydrofuran (15 mL) was added dropwise to the mixture. Stirring was continued at −78° C. for 1 h. A solution of 2-nitrobenzaldehyde (3.90 g, 25.8 mmoles) in tetrahydrofuran (20 mL) was then added to the mixture dropwise, and then stirring was continued at −78° C. for a further 2.5 h. The reaction was quenched with cold aqueous ammonium chloride and then diluted with water. The mixture was extracted twice with ethyl acetate and the aqueous phase was discarded.

The material was dried (magnesium sulfate) filtered, and concentrated to dryness. Silica gel chromatography afforded the desired product in 94% yield as light yellow foam. MS m/e (M-C₄H₈+H)⁺=353.1.

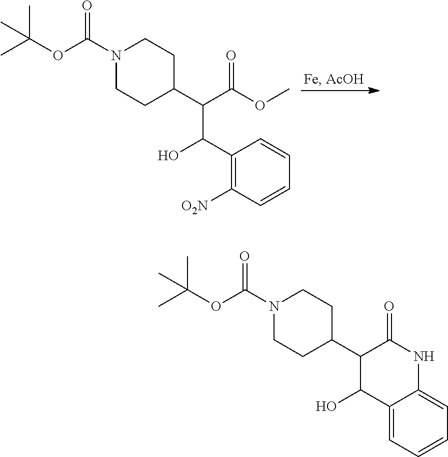 [SMILES: COC(=O)C(C1CCN(C(=O)OC(C)(C)C)CC1)C(O)c1ccccc1[N+](=O)[O-] ; CC(C)(C)OC(=O)N1CCC(C2C(=O)Nc3ccccc3C2O)CC1]

4-(4-Hydroxy-2-oxo-1, 2, 3, 4-tetrahydro-quinolin-3-yl)-piperidine-1-carboxylic acid tert-butyl ester. In a 3 neck flask fitted with a nitrogen inlet, thermometer, and a mechanical stirrer, 4-[2-hydroxy-1-methoxycarbonyl-2-(2-nitro-phenyl)-ethyl]-piperidine-1-carboxylic acid tert-butyl ester (9.93 g, 24.3 mmoles) was dissolved in acetic acid (1.75 moles, 100 mL). Iron powder (8.90 g, 159 mmoles) was added to the vessel with stirring. The stirred mixture was slowly heated to 80° C. for 30 min and then cooled to room temperature. It was then diluted with ethyl acetate and filtered through a pad of celite. Solids were washed with 20% methanol/ethyl acetate, and then with methanol. The filtrate was concentrated and the residue partitioned between ethyl acetate and aqueous sodium bicarbonate. The layers were separated. The resulting aqueous phase was extracted twice with ethyl acetate. The organic layers were combined. The mixture was washed twice with water and the aqueous phase was discarded. The material was dried (magnesium sulfate) filtered, and concentrated to dryness. Silica gel chromatography afforded the title compound as light yellow foam in 77% yield. MS m/e (M−H)⁻=345.1.

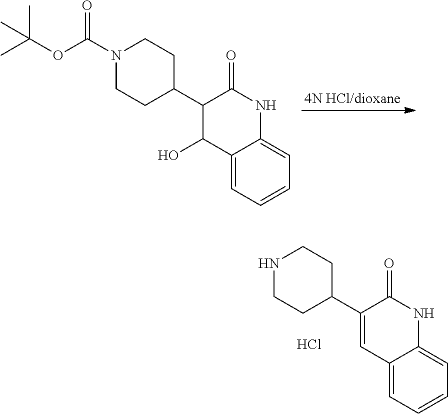 [SMILES: CC(C)(C)OC(=O)N1CCC(C2C(=O)Nc3ccccc3C2O)CC1 ; O=c1[nH]c2ccccc2cc1C1CCNCC1 ; Cl]

3-(Piperidin-4-yl)quinolin-2(1H) hydrochloride. A stirred solution of 4-(4-hydroxy-2-oxo-1,2,3,4-tetrahydro-quinolin-3-yl)-piperidine-1-carboxylic acid tert-butyl ester (5.60 g, 16.2 mmoles) in ethyl acetate (70 mL) was treated with HCl in dioxane (4N, 40 mmoles, 10 mL). The mixture was stirred at room temperature for 45 min. More HCl in dioxane (4N, 120 mmoles, 30 mL) was then added and stirring was continued at room temperature for 16 h. The resulting solid was collected by filtration and washed with ethyl acetate. It was then suspended in 5% water-isopropanol (100 mL) and the mixture was warmed to reflux and stirred for 20 min. The mixture was cooled to room temperature and stirred at room temperature for 16 h. The solid was collected by filtration, washed with isopropanol, and dried under high vacuum. The title compound was obtained as white solid in 75% yield. ¹H-NMR (DMSO-d₆) δ 11.85 (s, 1H), 9.02 (bs, 1H), 8.88 (bs, 1H), 7.70 (t, J=3.81 Hz, 2H), 7.53-7.30 (d, J=8.24 Hz, 1H), 7.17 (t, J=7.48 Hz, 2H), 3.36 (d, J=12.51 Hz, 2H), 3.10-2.94 (m, 3H), 2.01 (d, J=13.43 Hz, 2H), 1.87-1.73 (m, 2H); MS m/e (M+H)⁺=229.0.

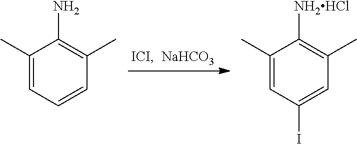

4-Iodo-2,6-dimethylbenzenamine hydrochloride. To a suspension of sodium bicarbonate (126 g, 1.5 moles) and 2,6-dimethylaniline (61.5 mL, 500 mmoles) in methanol (700 mL) was added iodine monochloride (1.0 M in dichloromethane, 550 mL, 550 mmoles) at room temperature over 1 h. After addition was complete, stirring was continued for 3 h. The reaction was filtered to remove excess sodium bicarbonate and the solvent removed in vacuo. The residue was re-dissolved in diethyl ether (1.5 L) and treated with hydrochloric acid (2M in ether, 375 mL, 750 mmoles). The resulting suspension was stored in the freezer (−15° C.) overnight. The solid was filtered and washed with diethyl ether until it became colorless, to give 126.5 g (89%) as a grey-green powder. ¹H-NMR (DMSO-d₆) δ 2.33 (s, 6H), 7.48 (s, 2H), 9.05 (bs, 3H); ¹³C-NMR (DMSO-d₆) δ 17.4, 91.5, 133.1, 131.2, 136.9.

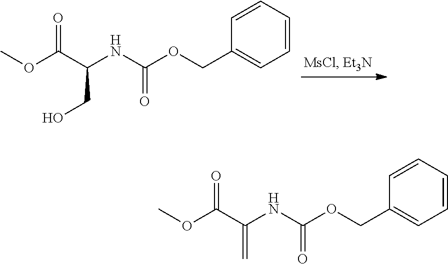 [SMILES: COC(=O)[C@H](CO)NC(=O)OCc1ccccc1 ; C=C(NC(=O)OCc1ccccc1)C(=O)OC]

Methyl 2-(benzyloxycarbonyl)acrylate. To a flame dried three-neck round bottom flask, fitted with a mechanical stirrer, was added (S)-methyl 2-(benzyloxycarbonyl)-3-hydroxypropanoate (129 g, 509 mmoles), anhydrous dichloromethane (2 L), and methanesulfonyl chloride (49.3 mL, 636 mmoles). The mixture was cooled to −15° C., and treated with triethylamine (213 mL, 1527 mmoles), dropwise, to ensure the temperature of the reaction mixture did not exceed 0° C. The addition of the first equivalent of triethylamine was exothermic. After addition of triethylamine, the mixture was stirred at 0° C. for 30 min. The cooling bath was removed and the mixture stirred at room temperature for 1.5 h. The reaction was quenched by addition of methanol (21 mL). The mixture was washed with 0.5% aqueous potassium bisulfate until the washings were pH 5, then saturated sodium bicarbonate, and brine, dried over sodium sulfate, and concentrated. Flash chromatography (silica gel, 1:9 ethyl acetate/hexanes) gave 111 g (92%) as a viscous colorless oil, which crystallized upon standing. ¹H-NMR (DMSO-d₆) δ 3.71 (s, 3H), 5.10 (s, 2H), 5.60 (s, 1H), 5.76 (s, 1H), 7.39-7.35 (m, 5H), 8.96 (s, 1H); ¹³C-NMR (DMSO-d₆) δ 52.3, 65.9, 127.8, 128.1, 128.3, 128.8, 133.3, 136.3, 153.5, 163.7.

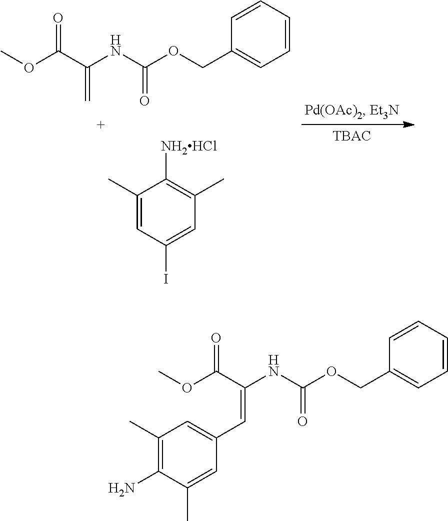

(Z)-Methyl 3-(4-amino-3,5-dimethylphenyl)-2-(benzyloxycarbonyl)acrylate. A 2 L round bottom flask was charged 4-iodo-2,6-dimethylbenzenamine hydrochloride salt (55 g, 194 mmoles), methyl 2-(benzyloxycarbonyl)acrylate (59.2 g, 252 mmoles), tetrabutylammonium chloride (59.2 g, 213 mmoles), palladium (II) acetate (4.34 g, 19.4 mmoles), and tetrahydrofuran (1.2 L, degassed by a flow of nitrogen for 30 min). The mixture was stirred so that a suspension was formed and then degassed by a flow of nitrogen for 30 min. Triethylamine (110 mL, 789 mmoles) was added and the resulting mixture was heated at reflux for 3 h. After cooling to room temperature, the reaction mixture was filtered through a pad of celite, washed with tetrahydrofuran (2×100 mL), and concentrated. The residue was dissolved in dichloromethane, washed with water (3×) and brine (2×), dried over sodium sulfate, and concentrated. Flash chromatography (silica gel, using 1:9 ethyl acetate/dichloromethane) gave a tan solid. The solid was recrystallized from warm methanol (210 mL) and water (100 mL). The mixture was held at room temperature overnight, then at 0° C. for 2 h, and finally at −15° C. for 2 h. The resulting solid was filtered, washed with ice cold 1:1 methanol/water, and dried under high vacuum overnight to give 44.7 g (65%) as a light tan solid which was a mixture of Z/E isomers (73:27). ¹H-NMR (DMSO-d₆) δ, 2.05 (s, 6H), 3.61 (s, 0.8 H), 3.68 (s, 2.2 H), 5.00 (s, 0.54 H), 5.13 (s, 1.46 H), 5.24 (s, 2H), 7.40-7.21 (m, 8H), 8.51 (s, 0.27 H), 8.79 (s, 0.73 H); ¹³C-NMR (DMSO-d₆) δ 17.8, 51.7, 65.3, 119.4, 120.0, 120.3, 127.3, 127.7, 128.3, 130.9, 135.8, 137.2, 146.9, 154.7, 166.0.

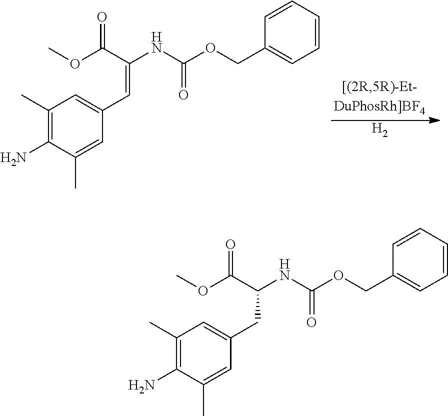 [SMILES: COC(=O)/C(=C\c1cc(C)c(N)c(C)c1)NC(=O)OCc1ccccc1 ; "H2" ; COC(=O)[C@@H](Cc1cc(C)c(N)c(C)c1)NC(=O)OCc1ccccc1]

(R)-Methyl 3-(4-amino-3,5-dimethylphenyl)-2-(benzyloxycarbonyl)propanoate. A flame-dried 2 L Parr hydrogenation bottle was charged with (Z)-methyl 3-(4-amino-3,5-dimethylphenyl)-2-(benzyloxycarbonyl)acrylate (84.5 g, 239 mmoles), dichloromethane (300 mL), and methanol (300 mL). The bottle was swirled so that a light brown suspension was formed. The mixture was degassed using a flow of nitrogen for 30 min. To this was quickly added (−)-1,2-bis((2R,5R)-2,5-diethylphospholano)-bezene(cyclooctadiene) rhodium (I) tetrafluoroborate ([(2R,5R)-Et-DuPhosRh]BF₄) (2.11 g, 3.20 mmoles). The bottle was immediately attached to a Parr Hydrogenator. After 5 cycles of hydrogen (60 psi) and vacuum, the bottle was pressurized to 65 psi and the suspension was agitated at room temperature for 16 h. The reaction had become homogeneous. The reaction mixture was concentrated, and the resulting residue purified by flash chromatography (silica gel, 1:9 ethyl acetate/dichloromethane) to give 82.9 g (98%). ¹H-NMR (DMSO-d₆) δ 2.04 (s, 6H), 2.65 (dd, J=13.4, 9.8 Hz, 1H), 2.82 (dd, J=13.7, 5.2 Hz, 1H), 3.62 (s, 3H), 4.15-4.10 (m, 1H), 4.41 (s, 2H), 5.00 (s, 2H), 6.68 (s, 2H), 7.37-7.28 (m, 5H), 7.70 (d, J=7.9 Hz, 1H); ¹³C-NMR (DMSO-d₆) δ 17.7, 35.9, 51.7, 56.1, 65.3, 120.4, 124.0, 127.5, 127.7, 128.2, 128.3, 136.9, 142.6, 155.9, 172.5.

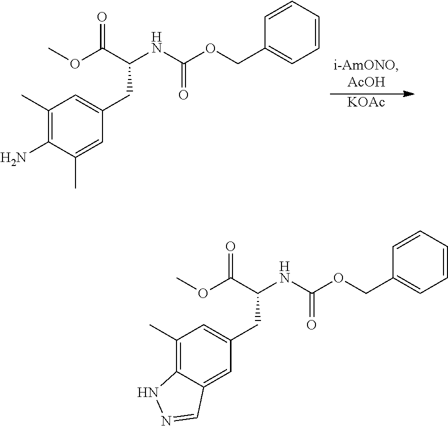 [SMILES: COC(=O)[C@@H](Cc1cc(C)c(N)c(C)c1)NC(=O)OCc1ccccc1 ; COC(=O)[C@@H](Cc1cc(C)c2[nH]ncc2c1)NC(=O)OCc1ccccc1 ; "KOAc"]

(R)-Methyl 2-(benzyloxycarbonyl)-3-(7-methyl-1H-indazol-5-yl)propanoate. (R)-Methyl 3-(4-amino-3,5-dimethylphenyl)-2-(benzyloxycarbonyl)propanoate (50.0 g, 140 mmoles) was weighed into a flame-dried 5 L three neck round bottom flask, followed by the addition of toluene (2.4 L) and glacial acetic acid (120 mL, 2.1 moles). The mixture was mechanically stirred to form a clear solution, and then potassium acetate (103 g, 1.05 moles) was added. To the resulting white suspension, iso-amyl nitrite (20.7 mL, 154 mmoles) was added dropwise at room temperature, and the resulting mixture was stirred at room temperature for 16 h. Saturated sodium bicarbonate (1 L) was added, followed by the careful addition of solid sodium bicarbonate to neutralize the acetic acid. The mixture was extracted with a mixture of dichloromethane (2 L) and brine (1.5 L). After separation, the aqueous layer was extracted with dichloromethane (500 mL). The combined organic layers were dried over anhydrous sodium sulfate and filtered. Solvents were removed to afford a tan solid, which was washed with hexanes (2 L) and toluene (150 mL). The solid was recrystallized from hot acetone (260 mL) and hexanes (700 mL). The slightly cloudy mixture was allowed to cool to room temperature slowly, then to 0° C. for 1.5 h, and finally to −15° C. for 1.5 h. The resulting solid was filtered and washed with ice-cold acetone/hexanes (1:1, 200 mL) to afford 39.1 g (76% yield). Analytical HPLC showed>98% UV purity. The enantiomeric excess (ee) was determined to be 99.8% (conditions: Chiralpak AD column, 4.6×250 mm, 10 μm; A=ethanol, B=0.05% diethylamine/heptane; 85% B @1.0 mL/min. for 55 min. The retention times for R was 44.6 min and for S was 28.8 min). ¹H-NMR (DMSO-d₆) δ 2.48 (s, 3H), 2.93 (dd, J=13.4, 10.7 Hz, 1H), 3.10 (dd, J=13.7, 4.9 Hz, 1H), 3.63 (s, 3H), 4.32-4.27 (m, 1H), 4.97 (s, 2H), 7.03 (s, 1H), 7.24-7.22 (m, 2H), 7.29-7.27 (m, 3H), 7.41 (s, 1H), 7.83 (d, J=8.2 Hz, 1H), 7.99 (s, 1H), 13.1 (s, 1H); ¹³C-NMR (DMSO-d₆) δ 16.7, 36.5, 51.8, 56.0, 65.3, 117.6, 119.6, 122.7, 127.2, 127.4, 127.6, 128.2, 129.3, 133.4, 136.8, 139.2, 155.9, 172.4. Mass spec.: 368.16 (MH)+.

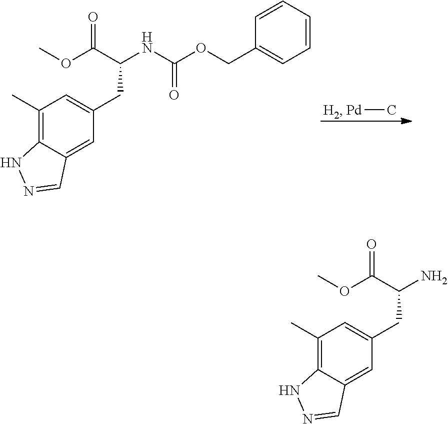

(R)-Methyl 2-amino-3-(7-methyl-1H-indazol-5-yl)propanoate. A Parr hydrogenation bottle was charged with (R)-methyl 2-(benzyloxycarbonyl)-3-(7-methyl-1H-indazol-5-yl)propanoate (11.0 g, 29.9 mmoles) and methanol (75 mL). The suspension was purged with nitrogen and treated with palladium (10% on charcoal, 700 mg). The bottle was shaken under hydrogen (15 psi) overnight. The mixture was filtered through a pad of celite to remove the catalyst. Concentration of the eluent gave 7.7 g (quant.) as an oil which was used without further purification. ¹H-NMR (CD₃OD) δ 2.54 (s, 3H), 2.98 (dd, J=13.5, 7.0 Hz, 1H), 3.09 (dd, J=13.5, 5.9 Hz, 1H), 3.68 (s, 3H), 3.75 (dd, J=7.0, 6.2 Hz, 1H), 7.01 (s, 1H), 7.39 (s, 1H), 7.98 (s, 1H). Mass spec.: 232.34 (M−H)⁻.

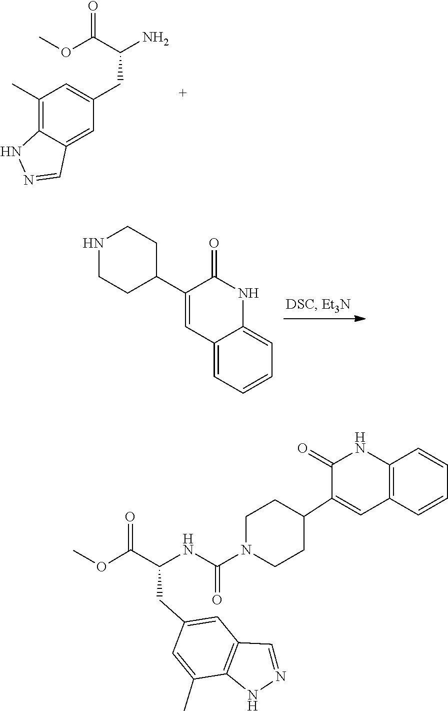 [SMILES: COC(=O)[C@H](N)Cc1cc(C)c2[nH]ncc2c1 ; O=c1[nH]c2ccccc2cc1C1CCNCC1 ; COC(=O)[C@@H](Cc1cc(C)c2[nH]ncc2c1)NC(=O)N1CCC(c2cc3ccccc3[nH]c2=O)CC1]

(R)-methyl 3-(7-methyl-1H-indazol-5-yl)-2-(4-(2-oxo-1,2-dihydroquinolin-3-yl)piperidine-1-carboxamido)propanoate. To a solution of (R)-methyl 2-amino-3-(7-methyl-1H-indazol-5-yl)propanoate hydrochloride (7.26 g, 27.0 mmoles) in dimethylformamide (50 mL) at room temperature was added N,N′-disuccinimidyl carbonate (7.60 g, 29.7 mmoles) followed by triethylamine (11.29 mL, 81 mmoles). The resulting mixture was stirred for 30 min and treated with 3-(piperidin-4-yl)quinolin-2(1H)-one (6.77 g, 29.9 mmoles) in portions. The reaction was allowed to stir for 24 h. The mixture was concentrated, dissolved in ethyl acetate, and washed sequentially with water, brine, and 0.5 N HCl (2×). The organic phase was dried over magnesium sulfate, filtered, and concentrated. The resulting residue was purified by flash chromatography (silica gel, 20:1 ethyl acetate/methanol) to give 11.9 g (78%). ¹H-NMR (CD₃OD) δ 13.0 (s, 1H), 11.8 (s, 1H), 7.98 (s, 1H), 7.63 (d, J=7.6 Hz, 1H), 7.57 (s, 1H), 7.45-7.41 (m, 2H), 7.27 (d, J=8.2 Hz, 1H), 7.16 (t, J=7.9 Hz, 1H), 7.03 (s, 1H), 6.85 (d, J=7.9 Hz, 1H), 4.31-4.26 (m, 1H), 4.10-4.08 (m, 2H), 3.60 (s, 3H), 3.07-3.01 (m, 2H), 2.93-2.88 (m, 1H), 2.77-2.67 (m, 2H), 2.48 (s, 3H), 1.78-1.72 (m, 2H), 1.34-1.26 (m, 2H). Mass spec.. 488.52 (MH)+.

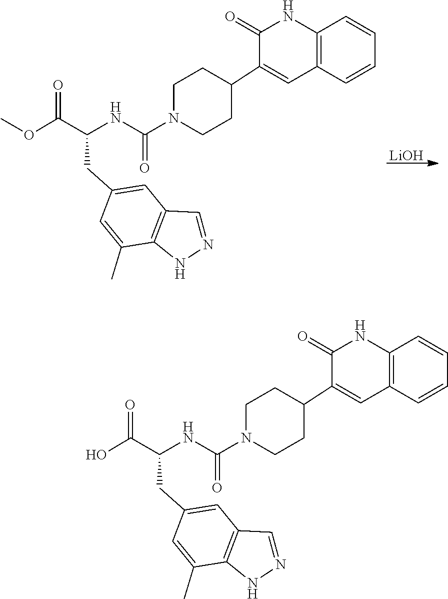 [SMILES: COC(=O)[C@@H](Cc1cc(C)c2[nH]ncc2c1)NC(=O)N1CCC(c2cc3ccccc3[nH]c2=O)CC1 ; "LiOH" ; Cc1cc(C[C@@H](NC(=O)N2CCC(c3cc4ccccc4[nH]c3=O)CC2)C(=O)O)cc2cn[nH]c12]

(R)-3-(7-methyl-1H-indazol-5-yl)-2-(4-(2-oxo-1,2-dihydroquinolin-3-yl)piperidine-1-carboxamido)propanoic acid. A solution of (R)-methyl 3-(7-methyl-1H-indazol-5-yl)-2-(4-(2-oxo-1,2-dihydroquinolin-3-yl)piperidine-1-carboxamido)propanoate_(5.50 g, 11.3 mmoles) in tetrahydrofuran (50 mL) and methanol (10 mL) was cooled to 0° C. To this was added a cold (0° C.) solution of lithium hydroxide monohydrate (0.95 g, 22.6 mmoles) in water (20 mL), dropwise over 15 min. The reaction was stirred at room temperature for additional 3 h. The mixture was concentrated to remove the organic solvents. The resulting residue was dissolved in a minimum amount of water, cooled to 0° C., and treated with cold (0° C.) 1N HCl until pH 2 was attained. The resulting solid was collected by filtration, washed with cold water and ether, and then dried overnight under high vacuum to give 5.0 g (94%) as a white solid. ¹H-NMR (DMSO-d₆) δ 13.05 (bs, 1H), 11.77 (s, 1H), 7.98 (s, 1H), 7.62 (d, J=8.0 Hz, 1H), 7.55 (s, 1H), 7.44 (d, J=8.2 Hz, 1H), 7.42 (s, 1H), 7.27 (d, J=8.2 Hz, 1H), 7.16 (t, J=7.6 Hz, 1H), 7.05 (s, 1H), 6.65 (d, J=7.9 Hz, 1H), 4.27-4.22 (m, 1H), 4.10-4.07 (m, 2H), 3.12-3.07 (m, 1H), 3.03-2.99 (m, 1H), 2.93-2.88 (m, 1H), 2.77-2.66 (m, 2H), 2.47 (s, 3H), 1.77-1.74 (m, 2H), 1.34-1.27 (m, 2H). Mass spec.: 474.30 (NM)*.

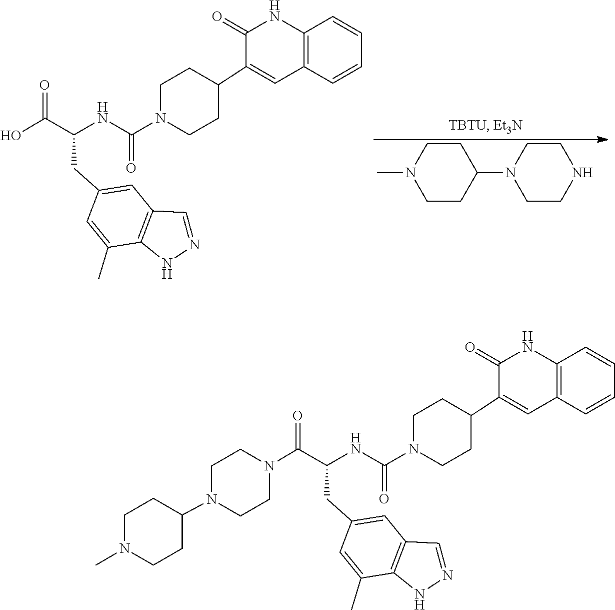 [SMILES: Cc1cc(C[C@@H](NC(=O)N2CCC(c3cc4ccccc4[nH]c3=O)CC2)C(=O)O)cc2cn[nH]c12 ; CN1CCC(N2CCNCC2)CC1 ; Cc1cc(C[C@@H](NC(=O)N2CCC(c3cc4ccccc4[nH]c3=O)CC2)C(=O)N2CCN(C3CCN(C)CC3)CC2)cc2cn[nH]c12]

(R)-N-(3-(7-methyl-1H-indazol-5-yl)-1-(4-(I-methylpiperidin-4-yl)piperazin-1-yl)-1-oxopropan-2-yl)-4-(2-oxo-1,2-dihydroquinolin-3-yl)piperidine-1-carboxamide (I). A flask was charged with (R)-3-(7-methyl-1H-indazol-5-yl)-2-(4-(2-oxo-1,2-dihydroquinolin-3-yl)piperidine-1-carboxamido)propanoic acid (2.9 g, 6.11 mmoles), triethylamine (3.00 mL, 21.5 mmoles), 1-(1-methylpiperidin-4-yl)piperazine (1.23 g, 6.72 mmoles), and dimethylformamide (10 mL). The resulting solution was treated with 2-(1H-benzotriazole-1-yl)-1,1,3,3-tetramethyluronium tetrafluoroborate (2.26 g, 7.03 mmoles) in portions. The reaction was allowed to stir at room temperature overnight. The mixture was concentrated under vacuum to remove dimethylformamide. The crude product was dissolved in 7% methanol in dichloromethane and purified by flash chromatography using 7% methanol in dichloromethane containing 2% of aqueous ammonium hydroxide as eluent. The pure fractions were collected and solvent was removed under vacuum. The desired product was crystallized from hot acetone to give the compound having Formula I in 77% yield. Analytical HPLC showed 99.0% UV purity at 230 nm. The enantiomeric excess (ee) was determined to be >99.9% (conditions: Chiralpak AD column, 4.6×250 mm, 10 μm; eluent: 70% (0.05% diethylamine)/heptane/30% ethanol; @1.0 mL/min. for 45 min. The retention times were 18.7 min for R and 28.1 min for S). ¹H-NMR (500 MHz, DMSO-d₆) δ ppm 13.01 (s, 1H), 11.76 (s, 1H), 7.96 (s, 1H), 7.62 (d, J=7.10 Hz, 1H), 7.60 (s, 1H), 7.42 (m, 1H), 7.36 (s, 1H), 7.26 (d, J=8.25 Hz, 1H), 7.14 (m, 1H), 7.00 (s, 1H), 6.69 (d, J=8.25 Hz, 1H), 4.78 (q, J=7.79 Hz, 1H), 4.14 (d, J=12.37 Hz, 2H), 3.54 (dd, J=9.16, 4.58 Hz, 1H), 3.24 (m, 1H), 3.11 (m, 1H), 2.97 (m, 1H), 2.89 (m, 2H), 2.69 (m, 4H), 2.32 (m, 1H), 2.21 (m, 1H), 2.07 (m, 4H), 1.95 (t, J=8.25 Hz, 1H), 1.87 (m, J=11.28, 11.28, 3.55, 3.44 Hz, 1H), 1.76 (t, J=12.03 Hz, 2H), 1.68 (t, J=11.11 Hz, 2H), 1.53 (t, J=8.25 Hz, 1H), 1.32 (m, 4H), 1.16 (m, 2H); ¹³C-NMR (DMSO-d₆) δ 16.80, 27.30, 30.51, 30.51, 30.67, 35.50, 38.04, 41.74, 44.00, 44.16, 45.35, 45.78, 48.14, 48.39, 51.45, 54.76, 54.76, 60.61, 114.53, 117.79, 119.29, 119.34, 121.57, 122.78, 127.46, 127.79, 129.29, 129.79, 133.31, 133.72, 136.98, 137.41, 139.12, 156.50, 161.50, 170.42. Accurate mass analysis: m/z 639.3770, [MH]⁺, Δ=−0.2 ppm. Optical rotation: −27.36° @ 589 nm, concentration=4.71 mg/mL in methanol.

### Description and Dosage Form

The physical and chemical properties of zavegepant (BHV-3500) drug substance mono-hydrochloride salt form are provided in Table 1.

TABLE 1
Physical and Chemical Properties

| Biohaven number | BHV-3500 |
|---|---|
| Molecular formula | C₃₆H₄₇ClN₈O₃ |
| Molecular weight | 675.26 (HCl salt); 638.82 (free base) |
| Appearance | White to off-white powder |
| Melting point | ~178° C. |
| pH-solubility profile | 105 mg/mL at pH = 8.2 and > 300 mg/mL |
| | at lower pH |
| pKa | 4.8 and 8.8 |
| logD | 1.21 |

The formulations used in the studies are provided in Table 2.

TABLE 2
Zavegepant Nasal Solution Proposed Formulations and Strengths - Deliverable Contents per Device

| | Concentration, mg/mL | | | | | | | |
|---|---|---|---|---|---|---|---|---|
| Ingredient | Placebo | 1.0 | 3.0 | 10.0 | 30.0 | 50.0 | 100.0 | 200.0 |
| BHV-3500 | 0 | 0.11 | 0.32 | 1.06 | 3.17 | 5.29 | 10.57 | 21.46 |
| (equivalent to free-base) | | (0.10) | (0.32) | (1.00) | (3.00) | (5.00) | (10.00) | (20.00) |

| | |
|---|---|
| Succinic acid, USP/NF | 0.59 |
| Dextrose monohydrate, USP/NF | 0.13 |
| NaOH 10N and/or HCl 1N | qs pH 6.0 ± 0.2 |
| Water for injection, USP | qs |
| To make | 100 μL |

### Intranasal Administration of Zavegepant

The drug product includes BHV-3500 compounded at 1 mg/mL to 200 mg/mL in 50 mM succinate solution and containing 1.25% (w/w) dextrose at pH 6.0 (preservative free). Manufacturing involves solubilizing excipients in a portion of the required Water for Injection (WFI) USP and adjusting the pH to 6.0+0.2 with sodium hydroxide or hydrochloric acid. The batch is brought to the target volume with WFI, sampled for bioburden, and filtered through a 0.22 μm filter to afford bioburden reduction.

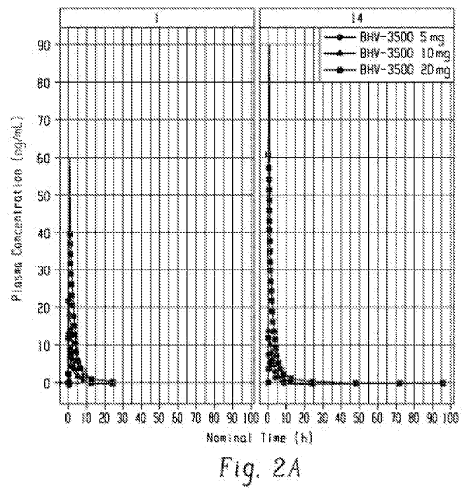

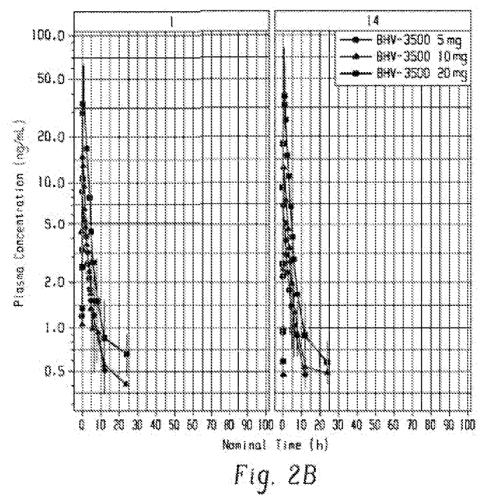

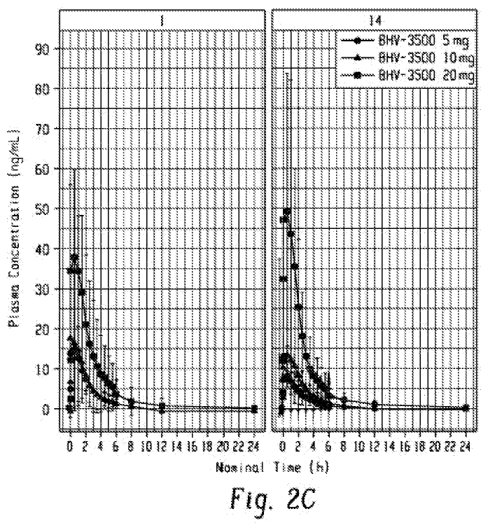

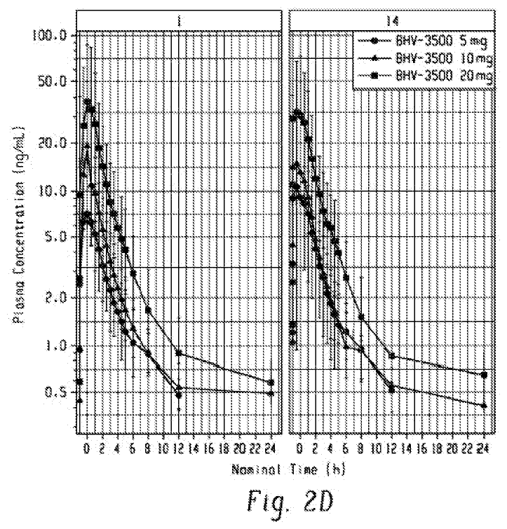

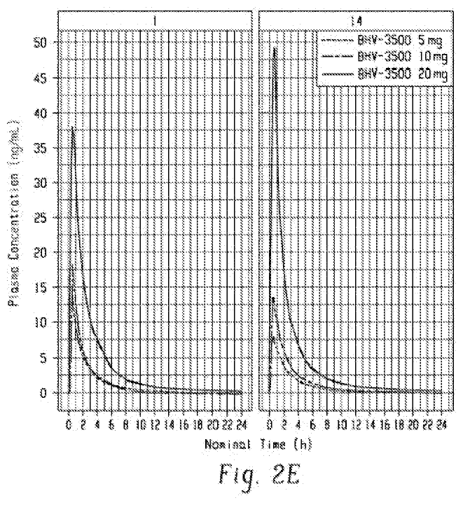

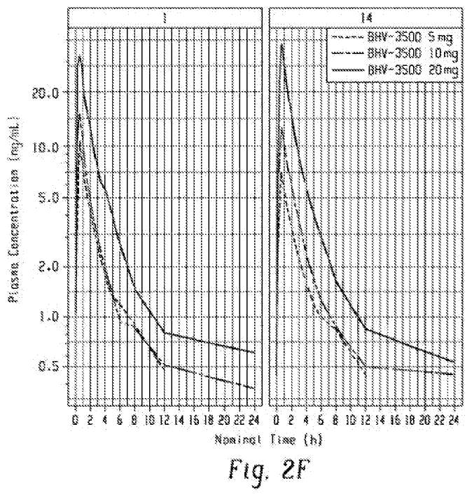

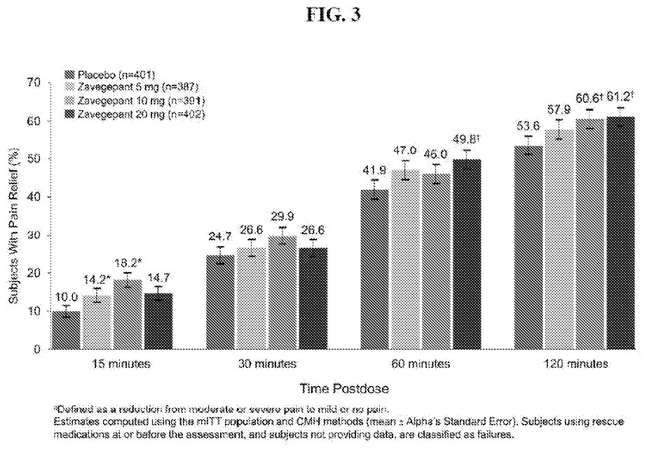

The filtered solution is then filled (125 μL) into Type 1 glass vials and sealed with rubber stoppers to deliver 100 μL of drug product. The sealed vials are then assembled into the Aptar Pharma UDS (Unidose System) device (FIG. 2) and then placed in appropriate secondary packaging. The device and secondary packaging are both labeled with information on one or both sides including study number, product name, strength, storage conditions, manufacturer and FDA required cautionary statements regarding investigational use and restricting access by children. For commercial product, each BHV-3500 product will be further packaged in a single blister with a peel off lid, which, in turn, will be packaged in other tertiary packaging (e.g., carton) for commercial distribution. FIG. 2A shows the Aptar Pharma UDS apparatus with a cross-section view (FIG. 2B) with location of all components. BHV-3500 nasal solution should be stored at 20° C. to 25° C. (68° F. to 77° F.) to designate room temperature storage. Excursions to 15° C. to 30° C. are permitted in the provided secondary packaging, protected from light.

BHV-3500 is a ready-to-use, unit dose, disposable nasal spray drug-device combination product. The device constituent part of the combination product includes a clear glass vial (Unit-Dose, Clear, USP Type I Glass Vial-sourced either from Nipro Glass or Ompi) with a rubber stopper (Black Chlorobutyl stopper, siliconized-sourced from West Pharmaceutical Services) (i.e., primary packaging components), assembled with an actuator subassembly (Subassembly of Polypropylene molded components with Steel cannula-sourced from Aptar Pharma) and a vial holder (Polypropylene molded component—sourced from Aptar Pharma) (i.e., secondary packaging components).

BHV-3500 nasal spray device consists of the following subassemblies and subcomponents:

Actuator ASM (Subassembly), which is Composed of:
Actuator (material of construction—Polypropylene—white color, sourced from Aptar Pharma)
Spray pin (Polypropylene—natural color—sourced from Aptar Pharma)
Cannula (Stainless Steel—natural color—sourced from Aptar Pharma)
Vial holder (Polypropylene—White color—sourced from Aptar Pharma)
Drug formulation filled Vial with Stopper
Glass vial—manufactured and supplied by following two vendors:
Nipro Glass, Germany AG
Nuova Ompi

The material of construction for vials from both suppliers is USP type I clear glass. The vials from both suppliers comply with the requirements set in USP 660: Glass Containers; USP 211: Arsenic; and USP 1660: Evaluation of the Inner Surface Durability of Glass Containers.
Rubber stopper—manufactured and supplied by West Pharmaceutical Services, Inc. The material of construction is chlorobutyl rubber (does not use natural rubber latex), and color is black. The stopper complies with the physiochemical tests as described in USP 381 “Elastomeric Closures for Injections”.

Actuator subassemblies are received as preassembled from Aptar Pharma. Both subassemblies and component are received and released by Renaissance (manufacturer for BHV-3500 product) based on vendor's Certificate of Conformity and incoming component inspections covering the visual appearance, identity and dimensional inspections, which provides assurance that all performance requirements are met.

### Clinical Pharmacology: Single Ascending Dose Study

BHV3500-101 is a completed Phase 1, single-center, placebo-controlled, randomized, double-blind, sequential SAD study. This study consisted of up to 11 cohorts. In each cohort, subjects were randomly assigned to receive either a single dose of zavegepant or placebo in a 3 to 1 ratio, for a total of 8 subjects. The primary objective of the study was to evaluate the safety and tolerability of zavegepant following IN administration of single ascending doses ranging from 0.1 mg to 40 mg, in healthy subjects. The secondary objectives were to characterize the PK profile of zavegepant following a single dose; identify maximum tolerated dose (MTD) of zavegepant if less than 40 mg; and describe the effect zavegepant on ECG parameters (i.e., QTc, PR interval, QRS complex, heart rate [HR], and T wave morphology).

BHV3500-101 was the first clinical study conducted with zavegepant to gather safety, tolerability, and PK information to support subsequent clinical studies with the compound. Zavegepant was administered using the Aptar Pharma UDS, a disposable device that delivers a single 100 μL spray. All subjects who received zavegepant 0.1 mg and 0.3 mg, as well as subject from the zavegepant 1 mg cohort were excluded from the PK analyses as these subjects had no detectable plasma concentrations (below lower limit of quantification [LLOQ]) at all time points measured. In total, 41 subjects were included in the PK analyses. A summary of the PK descriptive statistics is presented in Table 10 and an overview of the results is summarized below:
The administration of zavegepant as a single IN dose of 5 mg to 20 mg produced systemic exposures within the therapeutic range predicted to have efficacy from nonclinical models.
The rate and extent of absorption were greater for the 20 mg dose groups compared to the low and mid-dose groups (zavegepant 1 mg, 3 mg, 5 mg, and 10 mg). The rate and extent of absorption of the highest dose group (zavegepant 40 mg [2×20 mg]) was lower than the zavegepant 20 mg (1×20 mg) dose group.
Zavegepant was rapidly absorbed with peak zavegepant concentrations observed at 0.54 h following administration of a single IN dose (zavegepant 10 mg) with a median Tₘax ranging from 0.54 to 0.96 h across all doses and 0.54 to 0.77 h across the 5 mg to 20 mg range.
The median t_{1/2} of zavegepant ranged from 1.6 to 4.7 h across all doses, and from 2.5 to 4.4 h for 5 mg to 20 mg.
The mean residual area for zavegepant was less than 20% at all doses except the 1 mg (31.66%) indicating that a sampling period of 96 hours was sufficient to characterize the PK profile of zavegepant. This is equivalent to a mean AUC_{[0-4]} to AUC_{[0-inf]} ratio above 80%.

The results of the single ascending dose study are shown in Table 3.

TABLE 3
Summary Statistics for Zavegepant Pharmacokinetic Parameters
Following Intranasal Single Ascending Dose Administration

| | Intranasal Dose (100 μL^{a}) | | | | | | |
|---|---|---|---|---|---|---|---|
| | | | | | | 20 mg | 40 mg |
| | | | | | | (2 × 10 mg | (2 × 20 mg |
| Pharmacokinetic | 1 mg | 3 mg | 5 mg | 10 mg | 20 mg | sprays) | sprays) |
| Parameter | (n = 5) | (n = 6) | (n = 6) | (n = 6) | (n = 6) | (n = 6) | (n = 6) |
| Cₘₐₓ | 1.34 | 3.68 | 7.80 | 13.40 | 22.64 | 33.95 | 26.67 |
| (ng/mL) | (43.69) | (49.37) | (67.73) | (52.87) | (142.17) | 133.76 | (42.70) |
| GM (CV %) | | | | | | | |
| Tₘₐₓ | 0.96 | 0.78 | 0.60 | 0.54 | 0.77 | 0.55 | 0.59 |
| (h) Median | (0.37, 1.59) | (0.38, 1.59) | (0.56, 0.73) | (0.38, 0.59) | (0.38, 1.26) | (0.34, 1.29) | (0.36, 1.31) |
| (Min. Max) | | | | | | | |
| AUC [symbol] | 2.34 | 7.85 | 18.78 | 26.19 | 56.33 | 84.68 | 81.51 |
| (ng · h/mL) | (76.12) | (66.36) | (48.72) | (52.33) | (147.68) | (99.44) | (34.14) |
| GM (CV %) | | | | | | | |
| AUC_{[0-inf]} | 3.50 | 9.75 | 20.90 | 28.51 | 58.91 | 89.10 | 85.75 |
| (ng · h/mL) | (55.63) | (57.42) | (44.88) | (49.20) | (145.75) | (97.04) | (32.50) |
| GM (CV %) | | | | | | | |
| t_{1/2 el} | 1.64 | 2.24 | 2.36 | 2.92 | 2.39 | 4.03 | 4.40 |
| (h) Median | (0.72, 2.37) | (1.50, 2.86) | (2.24, 3.32) | (2.29, 3.66) | (1.81, 5.00) | (2.58, 6.77) | (2.76, 7.22) |
| (Min, Max) | | | | | | | |
| Residual Area | 31.66 | 18.90 | 10.04 | 8.08 | 4.38 | 4.93 | 4.93 |
| (%) | (50.24) | (56.27) | (39.35) | (37.99) | (42.69) | (51.83) | (35.39) |
| Mean (CV %) | | | | | | | |
| Kₑₗ | 0.52 | 0.33 | 0.28 | 0.25 | 0.28 | 0.18 | 0.17 |
| (1/h) | (51.57) | (23.36) | (13.50) | (16.38) | (30.66) | (35.23) | (38.13) |
| Mean (CV %) | | | | | | | |

^{a}Aptar Pharma UDS device [symbol]
indicates data missing or illegible when filed

### Clinical Pharmacology: Multiple Ascending Dose Study

A Phase 1, single-center, randomized, double-blind, placebo-controlled, sequential multiple ascending dose (MAD) study with 2 alternate dosing arms was conducted. Zavegepant (and placebo) was administered using the Aptar Pharma UDS, a disposable device that delivers a single 100 μL spray. The MAD portion of the study consists of 4 cohorts, with a maximum dose of 20 mg administered once daily for up to 14 days in 3 cohorts, and 20 mg administered twice daily for up to 8 days in the fourth cohort.

In addition to the 4 MAD cohorts, there were 2 alternate dosing cohorts, each consisting of 1 day dosing. The first alternate dosing cohort assessed the effect of 2 sequential administrations of 20 mg (20 mg spray [100 μL of 200 mg/mL] in alternate nostrils); with a 30 minute interval between administrations. A 2^{nd} alternate dosing cohort assessed the effect of 2 sequential administrations of 20 mg (20 mg spray [100 μL of 200 mg/mL] in alternate nostrils); with a nose blow and 5 minute interval between administrations.

The PK data were collected from subjects in Cohorts 1 to 4. All 36 subjects who received zavegepant in Cohorts 1 to 4 were included in the PK analyses. A summary of the PK descriptive statistics is presented in Table 4 and the results are summarized below:
Following single dose IN administration of zavegepant (Day 1), the geometric mean of Cₘₐₓ is 11.37, 16.31 and 34.71 ng/mL, respectively for the 5, 10 and 20 mg dose level. The geometric mean of AUC₀₋₂₄ is 24.95, 29.61 and 80.09 ng·h/mL, respectively.
Following multiple dose IN administration of zavegepant (Day 14), the geometric mean of Cₘₐₓ is 7.58, 12.98 and 40.93 ng/mL, respectively for the 5, 10 and 20 mg dose level. The geometric mean of AUCₜₐᵤ is 20.66, 32.85 and 90.98 ng·h/mL, respectively.
Tₘₐₓ occurred approximately 30 minutes after IN administration independent of the dose administered.
Mean elimination half-life ranged between 3.69 and 4.93 hours and tended to increase with dose.
All urine concentrations of zavegepant were below the limit of quantitation for all samples in Cohorts 1 to 3.

No to minimal accumulation of zavegepant was observed over the dose levels studied (ratios Day 14 vs Day 1 for Cₘₐₓ and AUC₀₋₂₄ are less than 2-fold, and ranged between 0.67 and 1.18).

The results of the multiple ascending dose studies are shown in Tables 4 and 5a to 5c.

TABLE 4
Summary Descriptive Statistics for Zavegepant Pharmacokinetic Parameters
Following Intranasal Multiple Ascending Dose Administration

| | Treatment | | | | | | | | | | | |
|---|---|---|---|---|---|---|---|---|---|---|---|---|
| | BHV-3500 5 mg | | | | BHV-3500 10 mg | | | | BHV-3500 20 mg | | | |
| | (Cohort 1) | | | | (Cohort 2) | | | | (Cohort 3) | | | |
| Variable | N | Mean* | SD | CV %* | N | Mean* | SD | CV %* | N | Mean* | SD | CV %* |

Day 1

| | | | | | | | | | | | | |
|---|---|---|---|---|---|---|---|---|---|---|---|---|
| AUC₀₋₂₄ | 9 | 24.95 | 21.88 | 104.32 | 9 | 29.61 | 31.51 | 77.28 | 9 | 80.09 | 76.55 | 85.25 |
| (ng*h/mL) | | | | | | | | | | | | |
| C [symbol] | 9 | 11.37 | 11.28 | 91.31 | 9 | 16.31 | 13.91 | 61.79 | 9 | 34.71 | 22.59 | 72.80 |
| (ng/mL) | | | | | | | | | | | | |
| Tₘₐₓ | 9 | 0.52 | 0.19 | 37.51 | 9 | 0.44 | 0.14 | 32.55 | 9 | 0.57 | 0.22 | 38.72 |
| (h) | | | | | | | | | | | | |

Day 14

| | | | | | | | | | | | | |
|---|---|---|---|---|---|---|---|---|---|---|---|---|
| AUC [symbol] | 8 | 20.66 | 15.35 | 75.50 | 9 | 32.85 | 18.32 | 53.96 | 9 | 90.98 | 89.45 | 69.05 |
| (ng*h/mL) | | | | | | | | | | | | |
| AUC [symbol] | 8 | 19.02 | 14.06 | 74.13 | 9 | 30.91 | 18.41 | 54.87 | 9 | 87.91 | 90.56 | 72.24 |
| (ng*h/mL) | | | | | | | | | | | | |
| AUC_{0-inf} | 8 | 22.41 | 14.08 | 60.16 | 9 | 33.85 | 19.29 | 51.42 | 9 | 92.44 | 91.87 | 69.82 |
| (ng*h/mL) | | | | | | | | | | | | |
| Cₘₐₓ | 8 | 7.58 | 5.87 | 74.80 | 9 | 12.98 | 6.78 | 52.61 | 9 | 40.93 | 40.25 | 79.17 |
| (ng/mL) | | | | | | | | | | | | |
| Cₘᵢₙ | 8 | 0.00 | 0.00 | NC | 9 | 0.05 | 0.16 | 300.00 | 9 | 0.20 | 0.31 | 158.02 |
| (ng/mL) | | | | | | | | | | | | |
| C_{avg} | 8 | 1.04 | 0.64 | 61.72 | 9 | 1.53 | 0.76 | 49.95 | 9 | 4.63 | 3.73 | 80.45 |
| (ng/mL) | | | | | | | | | | | | |
| F [symbol] % | 8 | 921.40 | 295.40 | 32.06 | 9 | 961.21 | 185.42 | 19.29 | 9 | 1094.77 | 226.75 | 20.71 |
| (%) | | | | | | | | | | | | |
| Tₘₐₓ | 8 | 0.52 | 0.23 | 43.43 | 9 | 0.57 | 0.12 | 21.09 | 9 | 0.56 | 0.12 | 21.25 |
| (h) | | | | | | | | | | | | |
| T_{1/2 el} | 8 | 3.69 | 0.28 | 7.47 | 9 | 3.84 | 1.61 | 42.00 | 9 | 4.93 | 2.07 | 41.89 |
| (h) | | | | | | | | | | | | |
| Kₑₗ | 8 | 0.19 | 0.01 | 7.40 | 9 | 0.20 | 0.06 | 29.96 | 9 | 0.16 | 0.06 | 35.88 |
| (1/h) | | | | | | | | | | | | |
| C [symbol] | 8 | 293.27 | 188.72 | 64.35 | 9 | 341.09 | 176.38 | 51.71 | 9 | 254.40 | 129.48 | 50.89 |
| (L/h) | | | | | | | | | | | | |
| Vz/F | 8 | 1573.9 | 1073.24 | 68.19 | 9 | 1657.78 | 535.67 | 32.31 | 9 | 1741.37 | 1196.05 | 68.68 |
| (L) | | | | | | | | | | | | |

*Geometric mean and CV % were presented for AUC₀₋₂₄, AUC [symbol] , AUC [symbol] , AUC [symbol] , and Cₘₐₓ [symbol]
indicates data missing or illegible when filed

TABLE 5a
Summary Statistics for Zavegepant Plasma Concentrations Following
Intranasal Multiple Ascending Dose Administration

| | | | | Nominal Time (h) | | | | | | | | | |
|---|---|---|---|---|---|---|---|---|---|---|---|---|---|
| | | | | 0.00 | 0.083 | 0.167 | 0.333 | 0.500 | 0.667 | 0.833 | 1.00 | 1.50 | 2.00 |
| Analyte | Treatment | Day | Statistics | Plasma Concentrations (ng/mL) | | | | | | | | | |
| BHV3500 | BHV-3500 | 1 | N | 9 | 9 | 9 | 9 | 9 | 9 | 9 | 9 | 9 | 9 |
| | 5 mg | | Mean | 0.00 | 0.89 | 4.95 | 12.49 | 13.80 | 12.15 | 10.84 | 9.14 | 6.94 | 5.39 |
| | | | SD | 0.00 | 0.84 | 4.36 | 11.10 | 11.06 | 9.08 | 7.48 | 6.12 | 4.83 | 3.87 |
| | | | CV % | NC | 94.22 | 88.12 | 88.89 | 80.10 | 74.70 | 68.98 | 66.98 | 69.53 | 71.81 |
| BHV3500 | BHV-3500 | 14 | N | 8 | 8 | 8 | 8 | 8 | 8 | 8 | 8 | 8 | 8 |
| | 5 mg | | Mean | 0.00 | [symbol] | 3.12 | 7.34 | 8.08 | 7.57 | 7.22 | 6.44 | 5.08 | 3.96 |
| | | | SD | 0.00 | [symbol] | 2.95 | 4.90 | 4.42 | 4.32 | 4.78 | 4.70 | 3.63 | 2.64 |
| | | | CV % | NC | 131.65 | 94.34 | 66.82 | 54.67 | 57.15 | 66.19 | 72.96 | 71.43 | 66.58 |
| BHV3500 | BHV-3500 | 1 | N | 9 | 9 | 9 | 9 | 9 | 9 | 9 | 9 | 9 | 9 |
| | 10 mg | | Mean | 0.00 | 0.80 | 5.18 | [symbol] | 18.25 | 16.49 | 14.65 | 11.65 | [symbol] | 5.85 |
| | | | SD | 0.00 | 0.77 | 2.47 | 9.01 | 13.94 | 14.18 | 12.91 | 10.88 | 6.98 | 4.74 |
| | | | CV % | NC | 96.85 | 47.63 | 55.56 | 76.39 | 85.97 | 88.14 | 93.43 | 85.04 | 80.94 |
| BHV3500 | BHV-3500 | 14 | N | 9 | 9 | 9 | 9 | 9 | 9 | 9 | 9 | 9 | 9 |
| | 10 mg | | Mean | 0.05 | 1.01 | 2.85 | 10.36 | 13.42 | 13.39 | 12.22 | 10.73 | 7.96 | 6.04 |
| | | | SD | 0.16 | 0.79 | 1.33 | 4.32 | 5.14 | 6.61 | 6.97 | 5.74 | 4.16 | 2.70 |
| | | | CV % | 300.00 | 78.54 | 46.68 | 41.65 | 38.28 | 49.38 | 57.04 | 53.49 | 52.28 | 44.80 |
| BHV3500 | BHV-3500 | 1 | N | 9 | 9 | 9 | 9 | 9 | 9 | 9 | 9 | 9 | 9 |
| | 20 mg | | Mean | 0.28 | 2.28 | 12.03 | 34.52 | [symbol] | 37.40 | 34.38 | 29.07 | 21.13 | 16.31 |
| | | | SD | 0.85 | 3.18 | 6.37 | 21.51 | 21.68 | 21.49 | 20.73 | 19.15 | 17.39 | 15.61 |
| | | | CV % | 300.00 | 139.63 | 52.92 | [symbol] | 57.01 | 57.44 | 60.30 | 65.89 | 82.32 | 95.72 |
| BHV3500 | BHV-3500 | 14 | N | 9 | 9 | 9 | 9 | 9 | 9 | 9 | 9 | 9 | 9 |
| | 20 mg | | Mean | 0.27 | 3.90 | 11.98 | 32.50 | 47.31 | 49.29 | 43.65 | 35.57 | 25.34 | 18.19 |
| | | | SD | 0.33 | 2.90 | 7.50 | 21.70 | 36.35 | 40.72 | 38.43 | 32.66 | 24.48 | 16.96 |
| | | | CV % | 124.16 | 74.24 | 62.60 | 66.76 | [symbol] | [symbol] | 88.04 | 91.83 | 96.60 | 93.21 |

[symbol]

indicates data missing or illegible when filed

TABLE 5b
Summary Statistics for Zavegepant Plasma Concentrations Following Intranasal Multiple Ascending Dose Administration

| | | | | Nominal Time (h) | | | | | | | | | |
|---|---|---|---|---|---|---|---|---|---|---|---|---|---|
| | | | | 2.50 | 3.00 | 3.50 | 4.00 | 4.50 | 5.00 | 6.00 | 8.00 | 12.0 | 24.0 |
| Analyte | Treatment | Day | Statistics | Plasma Concentrations (ng/mL) | | | | | | | | | |
| BHV3500 | BHV-3500 | 1 | N | 9 | 9 | 9 | 9 | 9 | 9 | 9 | 9 | 9 | 9 |
| | 5 mg | | Mean | 4.31 | 3.59 | 2.80 | [symbol] | 1.90 | 1.63 | 1.29 | 0.66 | 0.24 | 0.00 |
| | | | SD | 3.50 | 2.83 | 2.18 | 1.59 | 1.16 | 1.05 | 0.98 | 0.58 | 0.30 | 0.00 |
| | | | CV % | 81.17 | 78.94 | 77.87 | 68.81 | 61.08 | 64.04 | 75.99 | 88.82 | 125.58 | NC |
| BHV3500 | BHV-3500 | 14 | N | 8 | 8 | 8 | 8 | 8 | 8 | 8 | 8 | 8 | 8 |
| | 5 mg | | Mean | 3.13 | 2.64 | 2.19 | 1.91 | 1.62 | 1.39 | 1.16 | 0.57 | 0.31 | 0.00 |
| | | | SD | 1.94 | 1.62 | 1.36 | 1.13 | 0.89 | 0.73 | 0.58 | 0.51 | 0.27 | 0.00 |
| | | | CV % | 62.18 | 61.30 | 62.32 | 59.34 | 54.85 | 52.57 | 19.76 | 90.60 | 88.22 | NC |
| BHV3500 | BHV-3500 | 1 | N | 9 | 9 | 9 | 9 | 9 | 9 | 9 | 9 | 9 | 9 |
| | 10 mg | | Mean | 4.50 | 3.53 | 2.96 | 2.36 | 2.01 | 1.69 | 1.22 | 0.56 | 0.27 | 0.05 |
| | | | SD | 3.52 | 2.69 | 2.39 | 1.84 | 1.56 | 1.34 | 1.03 | 0.65 | 0.37 | 0.14 |
| | | | CV % | 78.35 | 76.07 | 80.83 | 78.09 | 77.38 | 79.05 | 84.48 | 116.26 | 138.62 | 300.00 |
| BHV3500 | BHV-3500 | 14 | N | 9 | 9 | 9 | 9 | 9 | 9 | 9 | 9 | 9 | 9 |
| | 10 mg | | Mean | 4.71 | 3.78 | 3.06 | 2.52 | 2.15 | 1.84 | 1.38 | 0.84 | 0.38 | 0.05 |
| | | | SD | 2.11 | 1.71 | 1.35 | 1.12 | 0.98 | 0.83 | 0.64 | 0.46 | 0.33 | 0.16 |
| | | | CV % | 44.82 | 45.13 | 44.18 | 44.38 | 45.57 | 45.09 | 45.92 | 54.78 | 87.84 | 300.00 |
| BHV3500 | BHV-3500 | 1 | N | 9 | 9 | 9 | 9 | 9 | 9 | 9 | 9 | 9 | 9 |
| | 20 mg | | Meon | 13.04 | 10.54 | 8.59 | 7.77 | 6.43 | 5.44 | 3.56 | 1.90 | 0.89 | 0.23 |
| | | | SD | 13.96 | 11.66 | 9.52 | 7.84 | 6.90 | 5.86 | 3.50 | 1.62 | 0.73 | 0.36 |
| | | | CV % | 107.03 | 110.55 | 110.86 | 100.85 | 107.45 | 107.74 | 98.28 | 85.24 | 82.14 | 158.48 |
| BHV3500 | BIIV-3500 | 14 | N | 9 | 9 | 9 | 9 | 9 | 9 | 9 | 9 | 9 | 9 |
| | 20 mg | | Mean | 13.11 | 10.03 | 8.32 | 7.02 | 5.94 | 4.97 | 3.39 | 2.04 | 1.03 | 0.27 |
| | | | SD | 10.70 | 7.82 | 6.27 | 5.71 | 4.54 | 3.69 | 2.37 | 1.73 | 0.71 | 0.35 |
| | | | CV % | 81.63 | 78.00 | 75.38 | 81.41 | 76.41 | 74.29 | 69.69 | 84.67 | 69.69 | 129.91 |

[symbol]

indicates data missing or illegible when filed

TABLE 5c
Summary Statistics for Zavegepant Plasma Concentrations Following
Intranasal Multiple Ascending Dose Administration

| | | | | Nominal Time (h) | | |
|---|---|---|---|---|---|---|
| | | | | 48.0 | 72.0 | 96.0 |
| | | | | Plasma Concentrations | | |
| Analyte | Treatment | Day | Statistics | (ng/mL) | | |
| BHV3500 | BHV-3500 | 1 | N | — | — | — |
| | 5 mg | | Mean | — | — | — |
| | | | SD | — | — | — |
| | | | CV % | — | — | — |
| BHV3500 | BHV-3500 | 14 | N | 8 | 8 | 8 |
| | 5 mg | | Mean | 0.00 | 0.00 | 0.00 |
| | | | SD | 0.00 | 0.00 | 0.00 |
| | | | CV % | NC | NC | NC |
| BHV3500 | BHV-3500 | 1 | N | — | — | — |
| | 10 mg | | Mean | — | — | — |
| | | | SD | — | — | — |
| | | | CV % | — | — | — |
| BHV3500 | BHV-3500 | 14 | N | — | — | — |
| | 10 mg | | Mean | 0.00 | 0.00 | 0.00 |
| | | | SD | 0.00 | 0.00 | 0.00 |
| | | | CV % | NC | NC | NC |
| BHV3500 | BHV-3500 | 1 | N | — | — | — |
| | 20 mg | | Mean | — | — | — |
| | | | SD | — | — | — |
| | | | CV % | — | — | — |
| BHV3500 | BHV-3500 | 14 | N | 9 | 9 | 9 |
| | 20 mg | | Mean | 0.00 | 0.00 | 0.00 |
| | | | SD | 0.00 | 0.00 | 0.00 |
| | | | CV % | NC | NC | NC |

NC—Not Calculated
The plasma concentration data are depicted in FIGS. 2A to 2F.

### Evaluation of BHV-3500 Efficacy and Safety

Efficacy and safety of intranasal zavegepant 5, 10 and 20 mg versus placebo were evaluated in randomized, dose ranging, placebo controlled, pivotal Phase 2/3 clinical trial (BHV3500-201, or Study 201), where 1,673 patients received acute treatment of migraine.

In Study 201, 5, 10, and 20 mg zavegepant administered as a single dose showed pain relief as early as 15 minutes post-dose using stratified CMH tests mITT subjects (Table 6, FIG. 3).

TABLE 6
Pain Relief at 15 Minutes Post-Dose Using Stratified CMH Tests mITT

| | | BHV-3500 | BHV-3500 | BHV-3500 | |
|---|---|---|---|---|---|
| Randomization | | 5 mg | 15 mg | 20 mg | Placebo |
| Stratum | Statistic | (N = [symbol] ) | (N = [symbol] ) | (N = 402) | (N = [symbol] ) |
| Overall | n/N ( [symbol] ) | 55/387 (14.2) | 71/ [symbol] ( [symbol] ) | [symbol] / [symbol] (14.7) | 40/401 ( [symbol] ) |
| | ASE | 1.8 | 1.9 | [symbol] | 1.5 |
| | ( [symbol] CI) | (10.0, [symbol] ) | ( [symbol] , [symbol] ) | [symbol] 10.5, [symbol] ) | ( [symbol] , [symbol] ) |
| | Stratified percentage | 4.2 | 8.2 | 4.7 | |
| | difference | | | | |
| | (BHV-3500 − Placebo) | | | | |
| | [symbol] | | | | |
| | ASE | 3.3 | 2.5 | [symbol] | |
| | (98.3 [symbol] CI) | (− [symbol] ) | [symbol] , 14.1) | ( [symbol] , [symbol] ) | |
| | P-value | 0.0673 | [symbol] | 0.0428 | |
| Prophylactic migraine | n/N ( [symbol] ) | [symbol] / [symbol] ( [symbol] ) | 13/50 (24.0) | [symbol] /57 ( [symbol] ) | 5/ [symbol] ( [symbol] ) |
| medication use: Yes | ASE | 4.3 | [symbol] | 5.6 | [symbol] |
| | (98.3 [symbol] CI) | ( [symbol] , 21.3) | ( [symbol] ) | (9.5, 38.1) | (0.0, 18.7) |
| | Percentage difference | 1.9 | 14.7 | 13.5 | |
| | (BHV-3500 − Placebo) | | | | |
| | ASE | 5.8 | 7.2 | [symbol] | |
| | ( [symbol] CI) | (−12.1, [symbol] ) | (−2.5, 32.0} | (−2.3, [symbol] ) | |
| | P-valve | 0.7503 | 0.0410 | 0.0465 | |
| Prophylactic migraine | n/N ( [symbol] ) | 49/ [symbol] (14.7) | 59/ [symbol] ( [symbol] ) | 46/345 (13.3) | 35/347 ( [symbol] ) |
| medication use: No | ASE | [symbol] | 2.0 | [symbol] | 1.6 |
| | (98.3 [symbol] CI) | (10.1, [symbol] ) | (12.4, 22.2} | ( [symbol] , 17.7} | (6.2, [symbol] ) |
| | Percentage difference | [symbol] | 7.2 | [symbol] | |
| | (BHV-3500 − Placebo) | | | | |
| | ASE | [symbol] | 2.6 | 2.4 | |
| | (98.3 [symbol] CI) | (−1.4, 10.7) | (1.0, 13.5) | ( [symbol] , [symbol] ) | |
| | P-value | [symbol] | 0.0057 | [symbol] | |

[symbol]

Stratified by prophylactic migraine medication use at randomization with Cochran-Mantel- [symbol] (CMH) weighting Subjects who [symbol] have missing data at 15 min post-dose (NC [symbol] F) or ( [symbol] ) took rescue medication at of before 15 min post-dose (R [symbol] F) are [symbol] as failures. [symbol]
indicates data missing or illegible when filed

Also, in Study 201, 10 and 20 mg zavegepant were statistically superior (p<0.05) to placebo on the co-primary endpoints of pain freedom and freedom from most bothersome symptom (MBS) at 2 hours using a single dose (Table 7). The benefits of zavegepant were durable and sustained without rescue medication out to 48 hours (nominal p<0.05), including: sustained pain freedom 2 to 24 hours (5, 10 and 20 mg); sustained pain freedom 2 to 48 (5, 10 and 20 mg); sustained pain relief 2 to 24 hours (5, 10 and 20 mg); sustained pain relief 2 to 48 (5 and 10 mg).

TABLE 7
Zavegepant met Co-Primary Endpoints of Pain Freedom
and Freedom from Most Bothersome Symptom

| | Zavegepant | | | |
|---|---|---|---|---|
| | 5 mg | 10 mg | 20 mg | Placebo |
| 2 Hour Endpoint | (N = 387) | (N = 391) | (N = 402) | (N = 401) |
| Pain Freedom | 19.6% | 22.5%* | 23.1%* | 15.5% |
| Freedom from MBS¹ | 39.0% | 41.9%* | 42.5%* | 33.7% |

¹Most Bothersome Symptom of photophobia, phonophobia or nausea
*p < 0.05

Zavegepant was also superior to placebo on multiple secondary endpoints demonstrating early activity (nominal p<0.05). Zavegepant showed rapid onset with pain relief at 15 minutes post-dose (10 and 20 mg), sustained pain relief from 2 to 24 hours post-dose (all three zavegepant groups), sustained pain freedom from 2 to 24 hours post-dose (all three zavegepant groups), sustained pain relief from 2 to 48 hours post-dose (zavegepant 5 mg and 10 mg), sustained freedom from 2 to 48 hours post-dose (all three zavegepant groups), and return to normal function as early as 30 minutes (20 mg). The 10 and 20 mg doses showed therapeutic benefits on both pain relief and return to normal function at 2 hours (Table 8, FIG. 3).

TABLE 8
Summary of Secondary Efficacy Endpoints - mITT Subjects

| | | Zavegepant | Zavegepant | Zavegepant | |
|---|---|---|---|---|---|
| | | 5 mg Group | 10 mg Group | 20 mg Group | Placebo |
| Secondary Endpoint | Statistic | (N = 387) | (N = 391) | (N = 402) | (N = 401) |
| (1) Pain relief at | n/N (%) | 224/387 (57.9) | 237/391 (60.6) | 246/402 (61.2) | 215/401 (53.6) |
| 2 hours postdose | (98.3% CI) | (51.9, 63.9) | (54.7, 66.5) | (55.4, 67.0) | (47.7, 59.6) |
| | Stratified Percentage | 4.2 | 7.1 | 7.5 | NA |
| | Difference | | | | |
| | (Zavegepant − Placebo)^{a} | | | | |
| | (98.3% CD) | (−4.2, 12.7) | (−1.3, 15.4) | (−0.8, 15.9) | NA |
| | p-value | 0.2296 | 0.0439 | 0.0302 | NA |
| (2) Return to normal | n/N (%) | 115/363 (31.7) | 122/354 (34.5) | 129/372 (34.7) | 101/369 (27.4) |
| function at | (98.3% CI) | (25.8, 37.5) | (28.4, 40.5) | (28.8, 40.6) | (21.8, 32.9) |
| 2 hours posidose^{b} | Stratified Percentage | 4.3 | 7.1 | 7.3 | NA |
| | Difference | | | | |
| | (Zavegepant − Placebo)^{a} | | | | |
| | (98.3% CI) | (−3.8, 12.3) | (−1.1, 15.3) | (−0.8, 15.4) | NA |
| | p-value | 0.2039 | 0.0389 | 0.0305 | NA |
| (3) Rescue medication | n/N (%) | 96/385 (24.9) | 101/388 (26.0) | 80/397 (20.2) | 109/400 (27.3) |
| use within | (98.3% CI) | (19.7, 30.2) | (20.7, 31.4) | (15.3, 25.0) | (21.9, 32.6) |
| 24 hours postdose^{c} | Stratified Percentage | −2.4 | −1.1 | −7.1 | NA |
| | Difference | | | | |
| | (Zavegepant − Placebo)^{a} | | | | |
| | (98.3% CI) | (−9.8, 5.1) | (−8.7, 6.4) | (−14.3, 0.0) | NA |
| | p-value | 0.4502 | 0.7154 | 0.0172 | NA |
| (4) Photophobia freedom | n/N (%) | 118/337 (35.0) | 121/340 (35.6) | 134/354 (37.9) | 109/358 (30.4) |
| at 2 hours | (98.3% CI) | (28.8, 41.2) | (29.4, 41.8) | (31.7, 44.0) | (24.6, 36.3) |
| postdose^{a} | Stratified Percentage | 4.6 | 5.1 | 7.4 | NA |
| | Difference | | | | |
| | (Zavegepant − Placebo)^{a} | | | | |
| | (98.3% CI) | (−3.9, 13.1) | (−3.4, 13.6) | (−1.0, 15.9) | NA |
| | p-value | 0.1986 | 0.1494 | 0.0352 | NA |
| (5) Phonophobia freedom | n/N (%) | 115/260 (44.2) | 107/239 (44.8) | 114/263 (43.3) | 94/276 (34.1) |
| at 2 hours postdose^{d} | (98.3% CI) | (36.9, 51.6) | (37.1, 52.5) | (36.0, 50.7) | (27.2, 40.9) |
| | Stratified Percentage | 10.1 | 10.8 | 9.3 | NA |
| | Difference | | | | |
| | (Zavegepant − Placebo)^{a} | | | | |
| | (98.3% CI) | (0.1, 20.1) | (0.6, 21.1) | (−0.6, 19.3) | NA |
| | p-value | 0.0161 | 0.0115 | 0.0249 | NA |
| (6) Pain relief at 60 | n/N (%) | 182/387 (47.0) | 180/391 (46.0) | 200/402 (49.8) | 168/401 (41.9) |
| minutes postdose | (98.3% CI) | (41.0, 53.1) | (40.0, 52.1) | (43.8, 55.7) | (36.0, 47.8) |
| | Stratified Percentage | 5.1 | 4.2 | 7.8 | NA |
| | Difference | | | | |
| | (Zavegepant − Placebo)^{a} | | | | |
| | (98.3% CI) | (−3.4, 13.5) | (−4.2, 12.6) | (−0.6, 16.2) | NA |
| | p-value | 0.1495 | 0.2274 | 0.0259 | NA |
| (7) Return to normal | n/N (%) | 82/363 (22.6) | 67/354 (18.9) | 70/372 (18.8) | 63/369 (17.1) |
| function at | (98.3% CI) | (17.3, 27.8) | (13.9, 23.9) | (14.0, 23.7) | (12.4, 21.8) |
| 60 minutes postdose^{b} | Stratified Percentage | 5.5 | 1.8 | 1.7 | NA |
| | Difference | | | | |
| | (Zavegepant − Placebo)^{a} | | | | |
| | (98.3% CI) | (−1.6, 12.5) | (−5.0, 8.7) | (−5.1, 8.4) | NA |
| | p-value | 0.0624 | 0.5222 | 0.5517 | NA |
| (8) Pain relief at 30 | n/N (%) | 103/387 (26.6) | 117/391 (29.9) | 107/402 (26.6) | 99/401 (24.7) |
| minutes postdose | (98.3% CI) | (21.2, 32.0) | (24.4, 35.5) | (21.3, 31.9) | (19.5, 29.8) |
| | Stratified Percentage | 1.9 | 5.3 | 1.9 | NA |
| | Difference | | | | |
| | (Zavegepant − Placebo)^{a} | | | | |
| | (98.3% CI) | (−5.5, 9.4) | (−2.3, 12.8) | (−5.5, 9.3) | NA |
| | p-value | 0.5359 | 0.0953 | 0.5398 | NA |
| (9) Return to normal | n/N (%) | 32/363 (8.8) | 27/354 (7.6) | 37/372 (9.9) | 20/369 (5.4) |
| function at | (98.3% CI) | (5.3, 12.4) | (4.2, 11.0) | (6.2, 13.7) | (2.6, 8.2) |
| 30 minutes postdose^{b} | Stratified Percentage | 3.4 | 2.1 | 4.5 | NA |
| | Difference | | | | |
| | (Zavegepant − Placebo)^{a} | | | | |
| | (98.3% CI) | (−1.2, 7.9) | (−2.3, 6.6) | (−0.2, 9.1) | NA |
| | p-value | 0.0753 | 0.2445 | 0.0216 | NA |
| (10) Sustained pain | n/N (%) | 169/387 (43.7) | 166/391 (42.5) | 179/402 (44.5) | 143/401 (35.7) |
| relief from 2 to | (98.3% CI) | (37.6, 49.7) | (36.5. 48.4) | (38.6, 50.5) | (29.9, 41.4) |
| 24 hours postdose | Stratified Percentage | 8.0 | 6.8 | 8.9 | NA |
| | Difference | | | | |
| | (Zavegepant − Placebo)^{a} | | | | |
| | (98.3% CI) | (−0.3, 16.4) | (−1.5, 15.1) | (0.7, 17.1) | NA |
| | p-value | 0.0205 | 0.0495 | 0.0098 | NA |
| (11) Sustained pain | n/N (%) | 55/387 (14.2) | 59/391 (15.1) | 63/402 (15.7) | 36/401 (9.0) |
| freedom from 2 to | (98.3% CI) | (10.0, 18.5) | (10.8, 19.4) | (11.3, 20.0) | (5.6, 12.4) |
| 24 hours postdose | Stratified Percentage | 5.3 | 6.1 | 6.7 | NA |
| | Difference | | | | |
| | (Zavegepant − Placebo)^{a} | | | | |
| | (98.3% CI) | (−0.2, 10.7) | (0.6, 11.6) | (1.2, 12.2) | NA |
| | p-value | 0.0210 | 0.0081 | 0.0036 | NA |
| (12) Sustained pain | n/N (%) | 155/387 (40.1) | 155/391 (39.6) | 156/402 (38.8) | 131/401 (32.7) |
| relief from 2 to | (98.3% CI) | (34.1, 46.0) | (33.7, 45.6) | (33.0, 44.6) | (27.1, 38.3) |
| 48 hours postdose | Stratified Percentage | 7.4 | 7.0 | 6.2 | NA |
| | Difference | | | | |
| | (Zavegepant − Placebo)^{a} | | | | |
| | (98.3% CI) | (−0.8, 15.6) | (−1.2, 15.1) | (−1.9, 14.2) | NA |
| | p-value | 0.0297 | 0.0404 | 0.0676 | NA |
| (13) Sustained pain | n/N (%) | 50/387 (12.9) | 54/391 (13.8) | 53/402 (13.2) | 30/401 (7.5) |
| freedom from 2 to | (98.3% CI) | (8.8, 17.0) | (9.6, 18.0) | (9.1, 17.2) | (4.3, 10.6) |
| 48 hours postdose | Stratified Percentage | 5.5 | 6.3 | 5.7 | NA |
| | Difference | | | | |
| | (Zavegepant − Placebo)^{a} | | | | |
| | (98.3% CI) | (0.3, 10.6) | (1.1, 11.6) | (0.6, 10.8) | NA |
| | p-value | 0.0111 | 0.0038 | 0.0075 | NA |
| (14) Nausca freedom | n/N (%) | 126/237 (53.2) | 131/243 (53.9) | 145/265 (54.7) | 122/239 (51.0) |
| at 2 hours | (98.3% CI) | (45.4, 60.9) | (46.3, 61.6) | (47.4, 62.0) | (43.3, 58.8) |
| postdose^{d} | Stratified Percentage | 1.8 | 2.9 | 3.7 | NA |
| | Difference | | | | |
| | (Zavegepant − Placebo)^{a} | | | | |
| | (98.3% CI) | (−9.2, 12.7) | (−8.0, 13.7) | (−7.0, 14.3) | NA |
| | p-value | 0.6987 | 0.5279 | 0.4092 | NA |
| (15) Pain relapse | n/N (%) | 24/76 (31.6) | 29/88 (33.0) | 35/93 (37.6) | 31/62 (50.0) |
| from 2 to 48 | (98.3% CI) | (18.8, 44.3) | (21.0, 45.0) | (25.6, 49.7) | (34.8, 65.2) |
| hours postdose^{e} | Stratified Percentage | −18.9 | −17.0 | −12.5 | NA |
| | Difference | | | | |
| | (Zavegepant − Placebo)^{a} | | | | |
| | (98.3% CI) | (−38.6, 0.9) | (−36.4, 2.5) | (−31.9, 7.0) | NA |
| | p-value | 0.0221 | 0.0366 | 0.1242 | NA |

Abbreviations: CI = confidence interval; NA = not applicable.
Subjects taking rescue medication at or before the time point are imputed as failures.
^{a}Stratified by prophylactic migraine medication use at randomization with CMH weighting.
^{b}Subjects with functional disability at on-study migraine attack onset.
^{c}Subjects with rescue medication start date ≤ study drug start date − 1 day and missing rescue medication start time are excluded.
^{d}Subjects with symptom present at on-study migraine attack onset.
^{e}Subjects with pain freedom at 2 hours postdosc.

Intranasal zavegepant was well tolerated and safe in this single dose trial. Individual adverse events (AEs) greater than 5% were: dysgeusia (13.5 to 16.1% in the zavegepant arms, and 3.5% in the placebo arm) and nasal discomfort (1.3 to 5.2% in the zavegepant arms, and 0.2% in the placebo arm). The majority (>80%) of AEs were mild in intensity, There was no signal of hepatoxicity as no subjects had AST or ALT>3× ULN, or total bilirubin>2× ULN, in any treatment arm (Table 9).

TABLE 9
Liver Function Test (LFT) Profile

| | Zavegepant | | | | |
|---|---|---|---|---|---|
| | 5 mg | 10 mg | 20 mg | All | Placebo |
| Measure¹ | (N = 388) | (N = 394) | (N = 403) | (N = 1185) | (N = 403) |
| ALT or AST > 3x ULN | 0 | 0 | 0 | 0 | 0 |
| Bilirubin > 2x ULN | 0 | 0 | 0 | 0 | 0 |

¹ALT alanine aminotransferase; AST aspartate aminotransferase; ULN Upper limit of normal

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

1-18. (canceled)

19. A method for treatment or prevention of a pain disorder associated with aberrant levels of CGRP in a subject in need thereof, the method comprising intranasally administering to the subject a composition comprising a therapeutically active component comprising a CGRP inhibitor.

20. The method according to claim 19, wherein the CGRP inhibitor is a CGRP antibody, a CGRP receptor antibody, an antigen-binding fragment from a CGRP antibody or a CGRP receptor antibody, a CGRP infusion inhibitory protein, a CGRP bio-neutralizing agent, a small molecule CGRP receptor antagonist, a small molecule CGRP inhibitor, or a polypeptide CGRP inhibitor.

21. The method according to claim 20, wherein the small molecule CGRP receptor antagonist is zavegepant, a solvate thereof, or a pharmaceutically acceptable salt thereof.

22. The method according to claim 21, wherein the intranasal administration of zavegepant at 10 mg as a single dose in the subjects results in at least 22.5% pain freedom at 2 hours after the dosing.

23. The method according to claim 21, wherein the intranasal administration of zavegepant at 20 mg as a single dose in the subjects results in at least 23.1% pain freedom at 2 hours after the dosing.

24. The method according to claim 21, wherein the intranasal administration of zavegepant at 10 mg as a single dose in the subjects results in at least 41.9% freedom from a most bothersome symptom of photophobia, phonophobia, or nausea at 2 hours after the dosing.

25. The method according to claim 21, wherein the intranasal administration of zavegepant at 10 mg as a single dose in the subjects results in at least 42.5% freedom from a most bothersome symptom of photophobia, phonophobia, or nausea at 2 hours after the dosing.

26. The method according to claim 21, wherein the intranasal administration of zavegepant at 10 mg or 20 mg is statistically superior to placebo on the co-primary endpoints of pain freedom and freedom from most bothersome symptom comprising photophobia, phonophobia, or nausea at 2 hours using a single dose.

27. The method according to claim 21, wherein the intranasal administration of zavegepant at 5 mg, 10 mg, or 20 mg in the subjects results in sustained pain freedom 2 to 48 hours after the dosing.

28. The method according to claim 21, wherein the intranasal administration of zavegepant at 5 mg, 10 mg, or 20 mg in the subjects results in sustained pain freedom 2 to 24 hours after the dosing.

29. (canceled)

30. The method according to claim 21, wherein the intranasal administration of zavegepant at 5 mg or 10 mg in the subjects results in sustained pain relief 2 to 48 hours after the dosing.

31. (canceled)

32. The method according to claim 19, wherein the condition is a disorder selected from chronic pain, neurogenic vasodilation, neurogenic inflammation, inflammatory pain, neuropathic pain, diabetic peripheral neuropathic pain, small fiber neuropathic pain, Morton's neuroma, chronic knee pain, chronic back pain, chronic hip pain, chronic finger pain, exercise-induced muscle pain, cancer pain, chronic inflammatory skin pain, pain from burns, pain from scars, complex regional pain syndrome, burning mouth syndrome, alcoholic polyneuropathy, chronic inflammatory demyelinating polyradiculoneuropathy, human immunodeficiency virus (HIV) or acquired immunodeficiency syndrome (AIDS)-associated neuropathy, drug-induced neuropathy, industrial neuropathy, lymphomatous neuropathy, myelomatous neuropathy, multi-focal motor neuropathy, chronic idiopathic sensory neuropathy, carcinomatous, neuropathy, acute pain autonomic neuropathy, compressive neuropathy, vasculitic/ischaemic neuropathy, tempero-mandibular joint pain, post-herpetic neuralgia, trigeminal neuralgia, chronic regional pain syndrome, eye pain, and tooth pain.

33. The method according to claim 19, wherein the condition is a disorder selected from non-insulin dependent diabetes mellitus, vascular disorders, inflammation, arthritis, thermal injury, circulatory shock, sepsis, alcohol withdrawal syndrome, opiate withdrawal syndrome, morphine tolerance, hot flashes in men and women, flushing associated with menopause, allergic dermatitis, psoriasis, encephalitis, ischaemia, stroke, epilepsy, neuroinflammatory disorders, neurodegenerative diseases, skin diseases, neurogenic cutaneous redness, skin rosaceousness, erythema, tinnitus, obesity, inflammatory bowel disease, irritable bowel syndrome, vulvodynia, polycystic ovarian syndrome, uterine fibroids, neurofibromatosis, hepatic fibrosis, renal fibrosis, focal segmental glomerulosclerosis, glomerulonephritis, IgA nephropathy, multiple myeloma, myasthenia gravis, Sjogren's syndrome, osteoarthritis, osteoarthritic degenerative disc disease, temporomandibular joint disorder, whiplash injury, rheumatoid arthritis, and interstitial cystitis.

34. The method according to claim 33, wherein the wherein, the condition is the skin disease and the skin disease is selected from recurrent herpes, contact hypersensitivity, prurigo nodularis, chronic pruritus, and uremic pruritus.

35. The method according to claim 19, wherein the condition is a disorder selected from chronic obstructive pulmonary disease, pulmonary fibrosis, bronchial hyperreactivity, asthma, cystic fibrosis, chronic idiopathic cough, and a toxic injury.

36. The method according to claim 35, wherein the toxic injury is selected from chlorine gas injury, mustard gas injury, acrolein injury, smoke injury, ozone injury, warfare chemical exposure, and industrial chemical exposure.

37-47. (canceled)

48. A method for treatment of vertigo, wherein the method comprises intranasally administering to the subject a therapeutically effective amount of a composition comprising a therapeutically active component comprising a CGRP inhibitor.

49. The method according to claim 48, wherein the CGRP inhibitor is a CGRP antibody, a CGRP receptor antibody, an antigen-binding fragment from a CGRP antibody or a CGRP receptor antibody, a CGRP infusion inhibitory protein, a CGRP bio-neutralizing agent, a small molecule CGRP receptor antagonist, a small molecule CGRP inhibitor, or a polypeptide CGRP inhibitor.

50. The method according to claim 49, wherein the small molecule CGRP receptor antagonist is zavegepant, a solvate thereof, or a pharmaceutically acceptable salt thereof.

51. The method according to claim 50, wherein the intranasal administration of zavegepant is administered as a single dose and at least one of
the intranasal administration of zavegepant at 5 mg as the single dose in the subjects result in at least 39.0% freedom from a most bothersome symptom of photophobia phonophobia, or nausea at 2 hours after the dosing;
the intranasal administration of zavegepant at 5 mg as the single dose in the subjects results in at least 14.2% pain freedom at 15 minutes after the dosing;
the intranasal administration of zavegepant at 5 mg as the single dose in the subjects results in at least 18.2% pain freedom at 15 minutes after the dosing;
the intranasal administration of zavegepant at 5 mg as the single dose in the subjects results in at least 14.7% pain freedom at 15 minutes after the dosing;
the intranasal administration of zavegepant at 5 mg as the single dose in the subjects results in at least 26.6% pain freedom at 30 minutes after the dosing;
the intranasal administration of zavegepant at 5 mg as the single dose in the subjects results in at least 29.9% pain freedom at 30 minutes after the dosing;
the intranasal administration of zavegepant at 5 mg as the single dose in the subjects results in at least 26.6% pain freedom at 30 minutes after the dosing;
the intranasal administration of zavegepant at 5 mg as the single dose in the subjects results in at least 47% pain freedom at 60 minutes after the dosing;
the intranasal administration of zavegepant at 5 mg as the single dose in the subjects results in at least 46.0% pain freedom at 60 minutes after the dosing;
the intranasal administration of zavegepant at 5 mg as the single dose in the subjects results in at least 49.8% pain freedom at 60 minutes after the dosing;
the intranasal administration of zavegepant at 5 mg as the single dose in the subjects results in at least 57.9% pain freedom at 120 minutes after the dosing;
the intranasal administration of zavegepant at 5 mg as the single dose in the subjects results in at least 60.6% pain freedom at 120 minutes after the dosing;
the intranasal administration of zavegepant at 5 mg as the single dose in the subjects results in at least 61.2% pain freedom at 120 minutes after the dosing.

52-64. (canceled)
